(19) United States
(12) Patent Application Publication
(10) Pub. No.: US 2012/0100130 A1
(43) Pub. Date: Apr. 26, 2012

(54) Novel Receptor Hetero-Dimers/-Oligomers

(75) Inventors: Kevin Donald George Pfleger, Nedlands (AU); Matthew Blake Dalrymple, Shenton Park (AU); James Williams, Como (AU); Elizabeth McCall, Dalkeith (AU)

(73) Assignee: DIMERIX BIOSCIENCE PTY LTD., Nedlands (AU)

(21) Appl. No.: 13/260,530

(22) PCT Filed: Mar. 26, 2010
(86) PCT No.: PCT/AU2010/000355

§ 371 Date: Jan. 11, 2012

(30) Foreign Application Priority Data
Mar. 27, 2009 (AU) 2009901336

(51) Int. Cl. A61K 39/395; G01N 33/567; C12N 5/071; C07K 19/00; C07K 16/42

(52) U.S. Cl. 424/131.1; 530/350; 530/387.2; 435/325; 435/317.1; 435/7.21

(57) ABSTRACT

A hetero-dimeric or hetero-oligomeric receptor, comprising at least one chemokine receptor subunit associated with at least one angiotensin receptor subunit.

### FIELD OF THE INVENTION

The present invention relates to a hetero-dimeric or hetero-oligomeric receptor, comprising at least one chemokine receptor subunit associated with at least one angiotensin receptor subunit.

### BACKGROUND ART

Proteins do not act in isolation in a cell, but in stable or transitory complexes, with protein-protein interactions being key determinants of protein function (Auerbach et al., (2002), Proteomics 2:611-623). Furthermore, proteins and protein complexes interact with other cellular components like DNA, RNA and small molecules. Understanding both the individual proteins involved in these interactions and their interactions are important for a better understanding of biological processes.

The primary physiological function of chemokine ligands (CCLs) reported by Allen (Allen, S. et al. (2007) Chemokine: Receptor Structure, Interactions and Antagonism. Annual Review Immunology 25:787-820) is the regulation of “cell migration during routine immune surveillance, inflammation and development”. CCLs are released in response to proinflammatory cytokines and selectively bind to a large family of G protein-coupled receptors, which mediate the physiological responses to chemokines. Chemokines were originally referred to as chemotactic cytokines.

Since discovering that the chemokine system plays an integral role in human immunodeficiency virus (HIV) infection and the pathogenesis of acquired immune deficiency syndrome (AIDS), considerable efforts have been made to understand the underlying mechanism(s) involved in order to develop potential intervention strategies (Lusso, P. (2006) HIV and the chemokine system: 10 years later. EMBO Journal 25:447-456). Furthermore, any deleterious immune response associated with a particular condition, including asthma, almost invariably result from a dysfunctional chemokine system. The pathogenesis of atherosclerosis has also been shown to involve chemokine signalling pathways, with the infiltration of macrophages into arterial lesions directly contributing to this aberrant inflammatory disorder (Boisvert, W. (2004) Modulation of Atherogenesis by Chemokines. Trends in Cardiovascular Medicine 14:161-165).

The renin-angiotensin system (RAS) plays an important role in the sympathetic nervous system and fluid homeostasis. Renin is a proteolytic enzyme secreted by the kidneys that mediates the formation of angiotensin I (AngI) from a globulin precursor, angiotensinogen (Rang, H. P., et al., Pharmacology: 3^{rd} Edition, 1995, Published by Churchill Livingstone, Edinburgh, UK.). AngI itself appears to have little physiological importance other than providing a substrate for a second enzyme, angiotensin-converting enzyme (ACE), which converts AngI to the highly active angiotensin II (AngII). However, it should be noted that AngII can be generated by alternative, ACE-independent mechanisms. AngII can in turn be metabolised to AngIII by aminopeptidases.

AngII is an extremely potent vasoconstrictor and as a consequence it has been extensively studied in the context of heart disease and hypertension pathogenesis (Ramasubbu, K. (2007) Anti-angiotensin Therapy: New Perspectives. Cardiology Clinics 25:573-580). In order to counter the deleterious vasoconstrictor effects of AngII in patients with hypertension, therapeutic strategies have been developed that intervene at the level of AngII signalling. In particular, compounds that inhibit the activity of ACE, preventing the conversion of AngI to AngII, and those that specifically block the activation of angiotensin receptors (ATRs), have been employed in the treatment of such conditions (Matchar, D. B. (2008) Systematic Review: Comparative Effectiveness of Angiotensin-Converting Enzyme Inhibitors and Angiotensin II Receptor Blockers for Treating Essential Hypertension. Annals of Internal Medicine 148:16-29).

The preceding discussion is intended only to facilitate an understanding of the invention. It should not be construed as in any way limiting the scope or application of the following description of the invention, nor should it be construed as an admission that any of the information discussed was within the common general knowledge of the person skilled in the appropriate art at the priority date.

### DISCLOSURE OF THE INVENTION

The inventors have discovered that the angiotensin receptor and the chemokine receptor associate. This has important implications regarding therapies for ailments associated with either receptor.

Recent studies have shown that GPCRs may not only act as monomers but also as homo- and hetero-dimers which causes altered ligand binding, signalling and endocytosis (Rios et al. (2000) Pharmacol. Ther. 92:71-87). The effect of drugs acting as agonists or antagonists of a specific receptor may therefore depend on the binding partners of this receptor. It may be desirable to limit the effect of a drug to a cellular response mediated by a specific receptor dimer. As Milligan (Milligan G. (2006), Drug Discovery Today 11:541-549) observes, while homo-dimerisation and -oligomerisation have limited implications for the drug discovery industry, “differential pharmacology, function and regulation of GCPR hetero-dimers and -oligomers suggest means to selectively target GPCRs in different tissues and hint that the mechanism of function of several pharmacological agents might be different in vivo than anticipated from simple ligand screening programmes that rely on heterologous expression of a single GPCR”.

The phrase “chemokine receptor” is to be understood to at least include the G protein-coupled CC chemokine receptors (CCRs), including: CC chemokine receptor 1 (CCR1), CC chemokine receptor 2 (CCR2), CC chemokine receptor 3 (CCR3), CC chemokine receptor 4 (CCR4), CC chemokine receptor 5 (CCR5), CC chemokine receptor 6 (CCR6), CC chemokine receptor 7 (CCR7), CC chemokine receptor 8 (CCR8), CC chemokine receptor 9 (CCR9), CC chemokine receptor 10 (CCR10). The phrase “chemokine receptor” is also to be understood to include the G protein-coupled CXC chemokine receptors (CXCRs), including: CXC chemokine receptor 1 (CXCR1), CXC chemokine receptor 2 (CXCR2), CXC chemokine receptor 3 (CXCR3), CXC chemokine receptor 4 (CXCR4), CXC chemokine receptor 5 (CXCR5), CXC chemokine receptor 6 (CXCR6) and CXC chemokine receptor 7 (CXCR7). The phrase “chemokine receptor” is to be also understood to mean the G protein-coupled XC chemokine receptor 1 (XCR1). The phrase “chemokine receptor” is to be further understood to include the G protein-coupled CX3 chemokine receptor (CX₃CR1). The phrase “chemokine receptor” is to be further understood to include the G protein-coupled CCX-CKR chemokine receptor (CCX-CKR). The phrase “chemokine receptor” is also understood to mean the G protein-coupled D6 chemokine receptor (D6). The phrase “chemokine receptor” is to be further understood to include the G protein-coupled DARC/Duffy chemokine receptor (DARC). This list of chemokine receptors is compiled from a review by Allen (Allen, S. et al. (2007) Chemokine: Receptor Structure, Interactions and Antagonism. Annual Review Immunology 25:787-820). Finally, the phrase “chemokine receptor” is to be further understood to include any newly discovered CCR/CXCR/XCR/CX₃CR/CCX-CKR/D6/DARC family members.

The phrase “angiotensin receptor” or “ATR” is to be understood to mean either angiotensin receptor 1 (AT1R; AT₁R) or angiotensin receptor 2 (AT2R; AT₂R), being G protein-coupled receptors analogous to those described by Porello et al. (Porello, E. R. (2008) The Angiotensin II Type 2 Receptor: An Enigma of Cardiovascular Pathophysiology. Frontiers in Bioscience. In Press), which are activated by angiotensin II (AngII) and/or angiotensin III (AngIII). “Angiotensin receptor” or “ATR” is to be further understood to include newly discovered angiotensin receptor family members.

In a first aspect of the invention, there is provided a hetero-dimeric or hetero-oligomeric receptor, comprising at least one chemokine receptor subunit associated with at least one angiotensin receptor subunit.

In a second aspect of the invention, there is provided a method for the treatment of a patient suffering from an angiotensin-related ailment by administering a therapeutically effective amount of chemokine receptor-related compound.

In one embodiment, the chemokine receptor-related compound is selective for the chemokine receptor relative to the angiotensin receptor.

In one embodiment, the chemokine receptor-related compound, may be co-administered with an angiotensin-related compound.

In a third aspect of the invention, there is provided a method for the treatment of a patient suffering from a chemokine-related ailment by administering a therapeutically effective amount of an angiotensin-related compound.

In one embodiment, the angiotensin receptor-related compound is selective for the angiotensin receptor relative to the chemokine receptor.

In one embodiment, the angiotensin receptor-related compound, may be co-administered with a chemokine receptor-related compound.

In a fourth aspect of the invention, there is provided a method for the manufacture of a medicament for the treatment of a patient suffering from an angiotensin-related ailment comprising use of a therapeutically effective amount of a chemokine receptor-related compound.

In one embodiment, the medicament may contain an angiotensin receptor-related compound.

In a fifth aspect of the invention, there is provided a method for the manufacture of a medicament for the treatment of a patient suffering from a chemokine-related ailment comprising use of a therapeutically effective amount of an angiotensin receptor-related compound.

In one embodiment, the medicament may contain a chemokine receptor-related compound.

In a sixth aspect of the invention, there is provided a method for the treatment of a patient suffering from an angiotensin-related ailment by administering a therapeutically effective amount of a chemokine-selective binding agent, or fragment thereof.

In a seventh aspect of the invention, there is provided a method for the treatment of a patient suffering from a chemokine-related ailment by administering a therapeutically effective amount of an angiotensin-selective binding agent, or fragment thereof.

In an eighth aspect of the invention, there is provided a method for the treatment of a patient suffering from a chemokine-related ailment or an angiotensin-related ailment comprising administering a therapeutically effective amount of a chemokine receptor/angiotensin receptor hetero-dimer/-oligomer selective agonist, selective inverse agonist, selective partial agonist, selective antagonist or other molecule that selectively interacts with the hetero-dimer/-oligomer, such as a selective allosteric modulator.

In a ninth aspect of the invention, there is provided the use of a therapeutically effective amount of a chemokine receptor/angiotensin receptor hetero-dimer/-oligomer selective agonist, selective inverse agonist, selective partial agonist, selective antagonist or other molecule that selectively interacts with the hetero-dimer/-oligomer, such as a selective allosteric modulator, for the manufacture of a medicament for the treatment of a patient suffering from a chemokine-related ailment or an angiotensin-related ailment.

In a tenth aspect of the invention, there is provided a method for screening a test compound for potential therapeutic activity against an angiotensin-related ailment using a detector capable of detecting changes in receptor activity, the method comprising the steps of:
a) exposing the test compound to a chemokine receptor;
b) assessing whether and/or the extent to which the activity of the chemokine receptor is modulated compared to activity of the chemokine receptor in the absence of the test compound;
said modulation being indicative of potential therapeutic activity against the angiotensin-related ailment.

In an eleventh aspect of the invention, there is provided a method for screening a test compound for potential therapeutic activity against a chemokine-related ailment using a detector capable of detecting changes in receptor activity, the method comprising the steps of:
a) exposing the test compound to an angiotensin receptor;
b) assessing whether and/or the extent to which the activity of the angiotensin receptor is modulated compared to activity of the angiotensin receptor in the absence of the test compound;
   said modulation being indicative of potential therapeutic activity against the chemokine-related ailment.

In a twelfth aspect of the invention, there is provided a method for screening a test compound for chemokine receptor/angiotensin receptor hetero-dimer/-oligomer selective activity using a detector capable of detecting changes in receptor activity, the method comprising the step of:
determining whether, and/or the extent to which, the test compound interacts with the angiotensin receptor while the angiotensin receptor is associated with the chemokine receptor compared to whether, and/or the extent to which the test compound interacts with the angiotensin receptor in the absence of the chemokine receptor;
   such that a test compound that exhibits greater affinity and/or potency and/or efficacy when interacting with the angiotensin receptor while the angiotensin receptor is associated with the chemokine receptor is selective for the chemokine receptor/angiotensin receptor hetero-dimer/-oligomer.

In a thirteenth aspect of the invention, there is provided a method for screening a test compound for chemokine receptor/angiotensin receptor hetero-dimer/-oligomer selective activity using a detector capable of detecting changes in receptor activity, the method comprising the steps of:
determining whether, and/or the extent to which, the test compound interacts with the chemokine receptor while the chemokine receptor is associated with the angiotensin receptor compared to whether, and/or the extent to which the test compound interacts with the chemokine receptor in the absence of the angiotensin receptor;
   such that a test compound that exhibits greater affinity and/or potency and/or efficacy when interacting with the chemokine receptor while the chemokine receptor is associated with the angiotensin receptor is selective for the chemokine receptor/angiotensin receptor hetero-dimer/-oligomer.

In a fourteenth aspect of the invention, there is provided a method for screening a test compound for chemokine receptor/angiotensin receptor hetero-dimer/-oligomer selective antagonism, or selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising the chemokine receptor;
   iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting a decrease in the signal as a determination of whether and/or the extent to which the test compound is an antagonist, partial agonist or negative allosteric modulator of the chemokine receptor/angiotensin receptor hetero-dimer/-oligomer, and optionally;
c) if the test compound is an antagonist, partial agonist or negative allsoteric modulator of the chemokine receptor/angiotensin receptor hetero-dimer/-oligomer, determining whether, or the extent to which the test compound is an antagonist, partial agonist or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

In a fifteenth aspect of the invention, there is provided a method for screening a test compound for chemokine receptor/angiotensin receptor hetero-dimer/-oligomer selective antagonism, selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising the angiotensin receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the angiotensin receptor;
b). detecting a decrease in the signal as a determination of whether and/or the extent to which the test compound is an antagonist, partial agonist or negative allosteric modulator of the chemokine receptor/angiotensin receptor hetero-dimer/-oligomer, and optionally;
c) if the test compound is an antagonist, partial agonist or negative allosteric modulator of the chemokine receptor/angiotensin receptor hetero-dimer/-oligomer, determining whether, or the extent to which the test compound is an antagonist, partial agonist or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

In a sixteenth aspect of the invention, there is provided a method for screening a test compound for chemokine receptor/angiotensin receptor hetero-dimer/-oligomer selective inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising a constitutively active chemokine receptor;
   wherein proximity of the first and second reporter components generates a signal; and wherein the modulator modulates the association of the interacting group with the chemokine receptor;
b) detecting a decrease in the signal as a determination of whether and/or the extent to which the test compound is an inverse agonist of the chemokine receptor/angiotensin receptor hetero-dimer/-oligomer, and optionally;
c) if the test compound is an inverse agonist of the chemokine receptor/angiotensin receptor hetero-dimer/-oligomer, determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine/angiotensin receptor hetero-dimer/-oligomer.

In a seventeenth aspect of the invention, there is provided a method for screening a test compound for chemokine receptor/angiotensin receptor hetero-dimer/-oligomer inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising a constitutively active angiotensin receptor;
   wherein proximity of the first and second reporter components generates a signal; and wherein the modulator modulates the association of the interacting group with the angiotensin receptor;
b) detecting a decrease in the signal as a determination of whether and/or the extent to which the test compound is an inverse agonist of the chemokine receptor/angiotensin receptor hetero-dimer/-oligomer, and optionally;
c) if the test compound is an inverse agonist of the chemokine receptor/angiotensin receptor hetero-dimer/-oligomer, determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine receptor/angiotensin receptor hetero-dimer/-oligomer.

In an eighteenth aspect of the invention, there is provided a method for screening a test compound for chemokine receptor/angiotensin receptor hetero-dimer/-oligomer selective positive allosteric modulation using a detector capable of detecting changes in receptor activity, the method comprising the steps of:
a) determining whether, and/or the extent to which, the test compound is a positive allosteric modulator of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising the chemokine receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the chemokine receptor;
b) detecting an increase in the signal as a determination of whether and/or the extent to which the test compound is a positive allosteric modulator of the chemokine receptor/angiotensin receptor hetero-dimer/-oligomer, and optionally;
c) determining whether and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine receptor/angiotensin receptor hetero-dimer/-oligomer.

In a nineteenth aspect of the invention, there is provided a method for screening a test compound for chemokine receptor/angiotensin receptor hetero-dimer/-oligomer selective positive allosteric modulation using a detector capable of detecting changes in receptor activity, the method comprising the steps of:
a) determining whether, and/or the extent to which, the test compound is a positive allosteric modulator of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising the angiotensin receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the angiotensin receptor;
b) detecting an increase in the signal as a determination of whether and/or the extent to which the test compound is a positive allosteric modulator of the chemokine receptor/angiotensin receptor hetero-dimer/-oligomer, and optionally;
c) determining whether, and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine receptor/angiotensin receptor hetero-dimer/-oligomer.

In the aforementioned methods of the invention, the step of determining whether, and/or the extent to which, the test compound interacts with the chemokine receptor while the chemokine receptor is associated with the angiotensin receptor; and/or the step of determining whether, and/or the extent to which, the test compound interacts with the angiotensin receptor while the angiotensin receptor is associated with the chemokine receptor may be performed by way of one or more of the methods described in the applicant's co-pending international patent application “Detection System and Uses Therefor” PCT/AU2007/001722 (published as WO 2008/055313).

In a twentieth aspect of the invention, there are provided selective agonists and/or selective antagonists and/or selective inverse agonists and/or selective allosteric modulators of the chemokine receptor/angiotensin receptor hetero-dimer/-oligomer.

In a twenty-first aspect of the invention, there is provided a cell, or fraction of a cell, in which both a chemokine receptor and an angiotensin receptor are over-expressed.

In a twenty-second aspect of the invention, there is provided a cell, or fraction of a cell, in which a chemokine receptor is over-expressed with an endogenously expressed angiotensin receptor.

In a twenty-third aspect of the invention, there is provided a cell, or fraction of a cell, in which an angiotensin receptor is over-expressed with an endogenously expressed chemokine receptor.

### BRIEF DESCRIPTION OF THE DRAWINGS

FIGS. 1 to 3 are illustrative of the technique by which the association of chemokine receptor and the angiotensin receptor was detected.

FIG. 1 shows the composition of the agents forming the basis of the system for detecting molecular associations: A first agent comprises a first interacting group coupled to a first reporter component; a second agent comprises a second interacting group coupled to a second reporter component; and a third agent comprises a third interacting group.

FIG. 2 shows how the administration of the modulator modulates the association of the second interacting group with the third interacting group, preferably by interacting with the third interacting group, either alone, or simultaneously with the first interacting group.

FIG. 3 shows that if the first and third interacting groups are associated, modulation of the association of the second and third interacting groups consequently modulates the proximity of the first and second reporter components thereby modulating the signal that is able to be detected by the detector. Therefore monitoring the signal generated by proximity of the first and second reporter components by the detector constitutes monitoring the association of the first and third agents. If the first and third interacting groups are not associated, the first and second reporter components will remain spatially separated and generation of a detectable signal is unlikely.

FIG. 4 shows the CC chemokine receptor 5 (CCR5) as IG1, Rluc8 as RC1, beta-arrestin 2 (barr2) as IG2, Venus as RC2 and hemagglutin epitope-tagged AT1R (HA-AT1R) as IG3. eBRET measurements at 37 C were carried out on HEK293FT cells transiently expressing CCR5/Rluc8 and barr2/Venus with HA-AT1R following treatment with either 10⁻⁷M macrophage inflammatory protein 1beta (MIP1β) or 10⁻⁶M angiotensin II (AngII) only or both MIP1β and AngII combined. In parallel, eBRET measurements at 37 C were carried out on HEK293FT cells transiently expressing CCR5/Rluc8 and barr2/Venus with pcDNA3 following treatment with 10⁻⁶M angiotensin II (AngII).

FIG. 5 shows the CXC chemokine receptor 2 (CXCR2) as IG1, Rluc8 as RC1, beta-arrestin 2 (barr2) as IG2, Venus as RC2 and hemagglutin epitope-tagged AT1R (HA-AT1R) as IG3. eBRET measurements at 37 C were carried out on HEK293FT cells transiently expressing CXCR2/Rluc8 and barr2/Venus with HA-AT1R following treatment with either 10⁻⁷M interleukin 8 (IL8) or 10⁻⁶M angiotensin II (AngII) only or both IL8 and AngII combined. In parallel, eBRET measurements at 37 C were carried out on HEK293FT cells transiently expressing CXCR2/Rluc8 and barr2/Venus with pcDNA3 following treatment with 10⁻⁶M angiotensin II (AngII).

FIG. 6 shows the CXC chemokine receptor 4 (CXCR4) as IG1, Rluc8 as RC1, beta-arrestin 2 (barr2) as IG2, Venus as RC2 and hemagglutin epitope-tagged AT1R (HA-AT1R) as IG3. eBRET measurements at 37 C were carried out on HEK293FT cells transiently expressing CXCR4/Rluc8 and barr2/Venus with HA-AT1R following treatment with either 10⁻⁷M stromal-cell derived factor-1α (SDF1α) or 10⁻⁶M angiotensin II (AngII) only or both SDF1α and AngII combined. In parallel, eBRET measurements at 37 C were carried out on HEK293FT cells transiently expressing CXCR4/Rluc8 and barr2/Venus with pcDNA3 following treatment with 10⁻⁶M angiotensin II (AngII).

FIG. 7 shows the angiotensin receptor 1 (AT1R) as IG1, Rluc8 as RC1, beta-arrestin 2 (barr2) as IG2, Venus as RC2 and CC chemokine receptor 2 (CCR2) as IG3. eBRET measurements at 37 C were carried out on HEK293FT cells transiently expressing AT1R/Rluc8 and barr2/Venus with CCR2 following treatment with either 10⁻⁷M monocyte chemotactic protein-1 (MCP1) or 10⁻⁶M angiotensin II (AngII). In parallel, eBRET signals were measured from cells transiently expressing AT1R/Rluc8 and barr2/Venus with pcDNA3 following treatment with 10⁻⁷M monocyte chemotactic protein-1 (MCP1).

FIG. 8 shows the angiotensin receptor 2 (AT2R) as IG1, Rluc8 as RC1, beta-arrestin 2 (barr2) as IG2, Venus as RC2 and CC chemokine receptor 2 (CCR2) as IG3. eBRET measurements at 37 C were carried out on HEK293FT cells transiently expressing AT2R/Rluc8 and barr2/Venus with CCR2 following treatment with either 10⁻⁷M monocyte chemotactic protein-1 (MCP1) or 10⁻⁶M angiotensin II (AngII). In parallel, eBRET signals were measured from cells transiently expressing AT2R/Rluc8 and barr2/Venus with pcDNA3 following treatment with either 10⁻⁷M monocyte chemotactic protein-1 (MCP1) or 10⁻⁶M angiotensin II (AngII).

### ABBREVIATIONS

ACE Angiotensin-converting enzyme
AIDS Acquired immune deficiency syndrome
AngI Angiotensin I peptide
AngII Angiotensin II peptide
AngIII Angiotensin III peptide
AT₁R Angiotensin receptor type 1
AT₂R Angiotensin receptor type 2
barr beta-arrestin.
BP Blood pressure
BRET Bioluminescence resonance energy transfer
CB Cannabinoid receptor
CCLs Chemokine ligands
CCRs CC Chemokine receptors
CXCRs CXC Chemokine receptors
CSF Cerebrospinal fluid
DOP Delta opioid.
eBRET extended BRET: BRET monitored over extended time periods.
ECFP Enhanced Cyan Fluorescent Protein, which is a variant of the Aequorea victoria green fluorescent protein gene (GFP).
EGFP Enhanced Green Fluorescent Protein is a red-shifted variant of wild-type GFP.
EYFP Enhanced Yellow Fluorescent Protein.
FRET Fluorescence resonance energy transfer.
GPCRs G-protein coupled receptors.
HA Hemagglutin epitope-tag.
His(6) Histidine tag consisting of 6 consecutive histidine residues.
HIV Human immunodeficiency virus
HPA Hypothalamic-pituitary-adrenal
IG Interacting group.
KOP Kappa opioid.
LPO Lateral preoptic area
mRFP1 Monomeric red fluorescent protein.
MCP1 Monocyte chemotactic protein-1
NPY Neuropeptide Y.
OR Opioid receptor.
PBS Phosphate-buffered saline.
pcDNA3 Eukaryotic expression vector.
PVN Paraventricular nucleus.
RC Reporter component.
REM Rapid eye movement.
RET Resonance energy transfer.
Rluc Renilla luciferase.
Rluc8 An improved Renilla luciferase.
SWS Slow wave sleep.
TYFP Topaz Yellow Fluorescent Protein.
Venus An improved Yellow Fluorescent Protein.
WT Wild type.

### BEST MODE(S) FOR CARRYING OUT THE INVENTION

### General

All publications, including patents and patent applications, cited herein, whether supra or infra, are hereby incorporated by reference in their entirety. However, publications mentioned herein are cited for the purpose of describing and disclosing the protocols, reagents and vectors that are reported in the publications and which may be used in connection with the invention. Nothing herein is to be construed as an admission that the invention is not entitled to antedate such disclosure by virtue of prior invention.

Furthermore, the practice of the present invention employs, unless otherwise indicated, conventional molecular biology, chemistry and fluorescence techniques, within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986; Lakowicz, J. R. Principles of Fluorescence Spectroscopy, New York: Plenum Press (1983) for fluorescence techniques.

As used herein and in the appended claims, the singular forms “a,” “an,” and “the” include the plural unless the context clearly dictates otherwise. Thus, for example, a reference to “a protein” includes a plurality of such proteins, and a reference to “an analyte” is a reference to one or more analytes, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The invention described herein may include one or more ranges of values (e.g. size, concentration etc). A range of values will be understood to include all values within the range, including the values defining the range, and values adjacent to the range that lead to the same or substantially the same outcome as the values immediately adjacent to that value which defines the boundary to the range.

Throughout this specification, unless the context requires otherwise, the word “comprise” or variations, such as “comprises” or “comprising” will be understood to imply the inclusion of a stated integer, or group of integers, but not the exclusion of any other integers or group of integers.

### Specific

As is apparent from the preceding summary of the invention, the invention relates, inter alia, to hetero-dimeric or hetero-oligomeric receptor, comprising at least one chemokine receptor subunit associated with at least one angiotensin receptor subunit. The terms “hetero-dimer” and “hetero-oligomer”, and variations such as “hetero-dimeric” and “hetero-oligomeric”, as used herein, refer to an entity within which at least one chemokine receptor is associated with at least one angiotensin receptor.

The phrase “associated with”, as used herein, refers to combination via any known direct or indirect stabilising atomic or molecular level interaction or any combination thereof, where the interactions include, without limitation, bonding interactions such as covalent bonding, ionic bonding, hydrogen bonding, co-ordinate bonding, or any other molecular bonding interaction, electrostatic interactions, polar or hydrophobic interactions, or any other classical or quantum mechanical stabilising atomic or molecular interaction.

Instances of different tissues having different repertoires of hetero-dimers have been reported. For example, 6′guanidinoaltrindole, an analogue of a well-known KOP receptor ligand, has been identified as a DOP-KOP hetero-dimer selective agonist, with efficacy as a spinally selective analgesic, leading to the conclusion that DOP-KOP heterodimers are expressed in the spinal cord, but not in the brain (Waldhoer, M. et al. (2005) A hetero-dimer selective agonist shows in vivo relevance of G-protein coupled receptor dimers. Proc. Natl. Acad. Sci. USA 102:9050-9055). Accordingly, the hetero-dimeric or hetero-oligomeric receptor, comprising at least one chemokine receptor subunit associated with at least one angiotensin receptor subunit represents a novel drug target.

As is the case with 6′guanidinoaltrindole, known ligands may exhibit differing abilities to trigger a hetero-dimeric receptor, which may uncover new applications for pre-existing molecules:
Hilairet S. et al. 2003 (J. Biol. Chem. 278:23731-23737) have recently shown that CB1 antagonists suppress appetite by acting through a CB1/OxR1 hetero-dimer pair.
It has been shown that somatostatin SSTR5 receptor will hetero-dimerise with a dopamine D2 receptor (Rocheville M. et al. (2000) Science 288:154-157).

As will be apparent from the following examples, the inventors herein have identified and characterised the molecular association of the chemokine receptor with the angiotensin receptor.

It will be apparent to a person skilled in the art that association of the chemokine receptor with the angiotensin receptor enables the use of compounds related to one receptor, including and without limitation, ligands of one receptor (be they agonists, inverse agonists or antagonists) in the treatment of ailments related to the other receptor.

Thus, the present invention encompasses a method for the treatment of a patient suffering from an angiotensin-related ailment by administering a therapeutically effective amount of a chemokine receptor-related compound.

The phrase “chemokine receptor-related compound” is to be understood to mean a compound that interacts with the chemokine receptor; or a compound that binds to a compound that interacts with the chemokine receptor, including but not limited to chemokines.

In one form of the invention, the chemokine receptor-related compound is a chemokine receptor agonist, inverse agonist or antagonist.

In one embodiment, the chemokine receptor-related compound is an allosteric modulator of the chemokine receptor.

In one embodiment, the chemokine receptor-related compound is a chemokine binding agent, or chemokine binding fragment thereof.

In one embodiment, the chemokine binding agent is an antibody, including a humanised antibody, a polyclonal antibody, a monoclonal antibody, a chimeric antibody, a CDR-grafted antibody and/or an anti-idiotypic antibody.

In one embodiment, the chemokine receptor-related compound is selective for the chemokine receptor relative to the angiotensin receptor. In one form of the invention, the chemokine receptor-related compound is selective for the chemokine receptor relative to the angiotensin receptor by a factor of at least 10. In one form of the invention, the chemokine receptor-related compound is selective for the chemokine receptor relative to the angiotensin receptor by a factor of at least 100. In one form of the invention, the chemokine receptor-related compound is selective for the chemokine receptor relative to the angiotensin receptor by a factor of at least 1000.

In the context of chemokine receptor-related compounds that are chemokine binding agents, or chemokine binding fragments thereof, the phrase “selective for the chemokine receptor relative to the angiotensin receptor” is to be understood to mean that the chemokine binding agent, or the chemokine binding fragment thereof, binds chemokines selectively relative to angiotensin.

In one embodiment, the chemokine receptor-related compound is co-administered with an angiotensin receptor-related compound.

The phrase “angiotensin receptor-related compound” is to be understood to mean a compound that interacts with the angiotensin receptor; a compound that binds to a compound that interacts with the angiotensin receptor, including but not limited to angiotensin; or a compound that modulates the production of a compound that interacts with the angiotensin receptor, including but not limited to angiotensin.

In one embodiment, the angiotensin receptor-related compound is an agonist, inverse agonist or antagonist of the angiotensin receptor.

In one embodiment, the angiotensin receptor-related compound is an allosteric modulator of the angiotensin receptor.

In one embodiment, the angiotensin receptor-related compound modulates the production of angiotensin.

In one embodiment, the angiotensin receptor-related compound is an angiotensin binding agent, or an angiotensin binding fragment thereof.

In one embodiment, the angiotensin binding agent is an antibody, including a humanised antibody, a polyclonal antibody, a monoclonal antibody, a chimeric antibody, a CDR-grafted antibody and/or an anti-idiotypic antibody.

The present invention further encompasses a method for the treatment of a patient suffering from a chemokine-related ailment by administering a therapeutically effective amount of an angiotensin receptor-related compound.

In one embodiment, the angiotensin receptor-related compound is an agonist, inverse agonist or antagonist of the angiotensin receptor.

In one embodiment, the angiotensin receptor-related compound is an allosteric modulator of the angiotensin receptor.

In one form of the invention, the angiotensin receptor-related compound is a compound that modulates the production of angiotensin.

In one form of the invention, the angiotensin receptor-related compound is an angiotensin binding agent, or an angiotensin binding fragment thereof.

In one embodiment, the angiotensin binding agent is an antibody, including a humanised antibody, a polyclonal antibody, a monoclonal antibody, a chimeric antibody, a CDR-grafted antibody and/or an anti-idiotypic antibody.

In one embodiment, the angiotensin receptor-related compound is selective for the angiotensin receptor relative to the chemokine receptor.

In one form of the invention, the angiotensin receptor-related compound is selective for the angiotensin receptor relative to the chemokine receptor by a factor of at least 10. In one form of the invention, the angiotensin receptor-related compound is selective for the angiotensin receptor relative to the chemokine receptor by a factor of at least 100. In one form of the invention, the angiotensin receptor-related compound is selective for the angiotensin receptor relative to the chemokine receptor by a factor of at least 1000.

In the context of angiotensin receptor-related compounds that modulate the production of a compound that interacts with the angiotensin receptor, the phrase selective for the angiotensin receptor relative to the chemokine receptor is to be understood to mean that the compound modulates the production of a compound that interacts with the angiotensin receptor to a greater extent than it modulates the production of chemokine.

In the context of angiotensin receptor-related compounds that are angiotensin binding agents, or angiotensin binding fragments thereof, the phrase selective for the angiotensin receptor relative to the chemokine receptor is to be understood to mean that the angiotensin binding agent, or the angiotensin binding fragment thereof, binds angiotensin selectively relative to chemokine.

In one embodiment, the angiotensin receptor-related compound is co-administered with a chemokine receptor-related compound.

In one form of the invention, the chemokine receptor-related compound is an agonist, inverse agonist or antagonist of the chemokine receptor.

In one embodiment, the chemokine receptor-related compound is an allosteric modulator of the chemokine receptor.

In one embodiment, the chemokine receptor-related compound is a chemokine binding agent, or a chemokine binding fragment thereof.

In one embodiment, the chemokine binding agent is an antibody, including a humanised antibody, a polyclonal antibody, a monoclonal antibody, a chimeric antibody, a CDR-grafted antibody and/or an anti-idiotypic antibody.

The present invention further encompasses a method for the manufacture of a medicament for the treatment of a patient suffering from an angiotensin-related ailment by administering a therapeutically effective amount of a chemokine receptor-related compound.

In one form of the invention, the chemokine receptor-related compound is an agonist, inverse agonist or antagonist of the chemokine receptor.

In one embodiment, the medicament contains an angiotensin receptor-related compound.

In one form of the invention, the angiotensin receptor-related compound is an agonist, inverse agonist or antagonist of the angiotensin receptor.

The present invention further encompasses a method for the manufacture of a medicament for the treatment of a patient suffering from a chemokine-related ailment by administering a therapeutically effective amount of an angiotensin receptor-related compound.

In one form of the invention, the angiotensin receptor-related compound is an agonist, inverse agonist or antagonist of the angiotensin receptor.

In one embodiment, the medicament contains a chemokine receptor-related compound.

In one form of the invention, the chemokine receptor-related compound is an agonist, inverse agonist or antagonist of the chemokine receptor.

Thus, the present invention encompasses a method for the treatment of a patient suffering from an angiotensin-related ailment by administering a therapeutically effective amount of a chemokine-selective binding agent, or fragment thereof.

The chemokine selective binding agent may be an antibody, including a humanised antibody, a polyclonal antibody, a monoclonal antibody, a chimeric antibody, a CDR-grafted antibody and/or an anti-idiotypic antibody.

The present invention further encompasses a method for the treatment of a patient suffering from a chemokine-related ailment by administering a therapeutically effective amount of an angiotensin-selective binding agent, or fragment thereof.

The angiotensin-selective binding agent may be an antibody, including a humanised antibody, a polyclonal antibody, a monoclonal antibody, a chimeric antibody, a CDR-grafted antibody and/or an anti-idiotypic antibody.

The present invention further encompasses a method for the treatment of a patient suffering from a chemokine-related ailment or an angiotensin-related ailment by administering a therapeutically effective amount of a chemokine receptor/angiotensin receptor hetero-dimer/-oligomer selective agonist, selective inverse agonist, selective partial agonist, selective antagonist or other molecule that selectively interacts with the hetero-dimer/-oligomer, such as a selective allosteric modulator.

The present invention further encompasses the use of a therapeutically effective amount of a chemokine receptor/angiotensin receptor hetero-dimer/-oligomer selective agonist, selective inverse agonist, selective antagonist or other molecule that selectively interacts with the hetero-dimer/-oligomer, such as a selective allosteric modulator, for the manufacture of a medicament for the treatment of a patient suffering from a chemokine-related ailment or an angiotensin-related ailment.

In one form of the invention the chemokine receptor/angiotensin receptor hetero-dimer/-oligomer selective agonist, inverse agonist, antagonist or other molecule that interacts with the hetero-dimer/-oligomer, such as an allosteric modulator is .selective for the chemokine receptor/angiotensin receptor hetero-dimer/-oligomer by a factor of at least 10. In one form of the invention, the chemokine receptor/angiotensin receptor hetero-dimer/-oligomer selective agonist, inverse agonist, partial agonist, antagonist or other molecule that interacts with the hetero-dimer/-oligomer, such as an allosteric modulator, is selective for the chemokine receptor/angiotensin receptor hetero-dimer/-oligomer by a factor of at least 100. In one form of the invention, the chemokine receptor/angiotensin receptor hetero-dimer/-oligomer selective agonist, inverse agonist, partial agonist, antagonist or other molecule that interacts with the hetero-dimer/-oligomer, such as an allosteric modulator, is selective for the chemokine receptor/angiotensin receptor hetero-dimer/-oligomer by a factor of at least 1000.

The present invention further encompasses a method for the treatment of a patient suffering from a chemokine-related ailment by administering a therapeutically effective amount of a selective angiotensin receptor/chemokine receptor hetero-dimer/-oligomer agonist, selective inverse agonist selective partial agonist, selective antagonist or other molecule that selectively interacts with the hetero-dimer/-oligomer, such as a selective allosteric modulator.

In one embodiment, the selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator is co-administered with a chemokine receptor-related compound.

In one embodiment, the chemokine receptor-related compound is a chemokine receptor agonist, inverse agonist or antagonist.

In one embodiment, the selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator is co-administered with an angiotensin receptor-related compound.

In one embodiment, the angiotensin receptor-related compound is an angiotensin receptor agonist, inverse agonist or antagonist.

The present invention further encompasses a method for the treatment of a patient suffering from an angiotensin-related ailment by administering a therapeutically effective amount of a selective angiotensin receptor/chemokine receptor hetero-dimer/-oligomer agonist, selective inverse agonist, selective partial agonist selective antagonist or other molecule that selectively interacts with the hetero-dimer/-oligomer, such as a selective allosteric modulator.

In one embodiment, the selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator is co-administered with a chemokine receptor-related compound.

In one embodiment, the chemokine receptor-related compound is a chemokine receptor agonist, inverse agonist or antagonist.

In one embodiment, the selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator is co-administered with an angiotensin receptor-related compound.

In one embodiment, the angiotensin receptor-related compound is an angiotensin agonist, inverse agonist or antagonist.

The present invention further encompasses a method for the manufacture of a medicament for the treatment of a patient suffering from a chemokine-related ailment comprising use of a therapeutically effective amount of a selective angiotensin receptor/chemokine receptor hetero-dimer/-oligomer agonist, inverse agonist or antagonist.

The present invention further encompasses a method for the manufacture of a medicament for the treatment of a patient suffering from a chemokine-related ailment comprising use of a therapeutically effective amount of a selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator.

In one embodiment, the medicament contains an angiotensin receptor-related compound. In one embodiment, the angiotensin receptor-related compound is an angiotensin agonist, inverse agonist or antagonist.

In one embodiment, the medicament contains a chemokine receptor-related compound. In one embodiment, the chemokine receptor-related compound is a chemokine receptor agonist, inverse agonist or antagonist.

The present invention further encompasses a method for the manufacture of a medicament for the treatment of a patient suffering from an angiotensin-related ailment comprising use of a therapeutically effective amount of a selective angiotensin receptor/chemokine receptor hetero-dimer/-oligomer agonist, inverse agonist or antagonist.

The present invention further encompasses a method for the manufacture of a medicament for the treatment of a patient suffering from an angiotensin-related ailment comprising use of a therapeutically effective amount of a selective angiotensin receptor/chemokine receptor hetero-dimer/-oligomer agonist, selective inverse agonist, selective partial agonist, selective antagonist or other molecule that selectively interacts with the hetero-dimer/-oligomer, such as a selective allosteric modulator.

In one embodiment, the medicament contains an angiotensin receptor-related compound. In one embodiment, the angiotensin-receptor-related compound is an angiotensin receptor agonist, inverse agonist or antagonist.

In one embodiment, the medicament contains a chemokine receptor-related compound. In one embodiment, the chemokine receptor-related compound is a chemokine receptor agonist, inverse agonist or antagonist.

Chemokine-related ailments include ailments that are related to increased or decreased production of chemokines, and/or increased or decreased responsiveness of cells to chemokines. A chemokine-related ailment should also be understood to mean a condition in which chemokine receptors display aberrant characteristics, are the target of a particular pathogen or are a target of a pharmacological intervention. The following list provides some examples of chemokine-related ailments:
HIV infection and AIDS pathogenesis
Ischemia and reperfusion injury;
Atherogenesis;
Chronic obstructive pulmonary disease;
Asthma and allergy;
Renal disease
Rheumatoid arthritis

However, it should be understood that the phrase chemokine-related interventions and the phrase a chemokine-related ailment is not limited thereto.

Known CC chemokine receptor-related compounds include; the CC chemokine ligand 1 (CCL1), CC chemokine ligand 2 (CCL2; also known as monocyte chemotactic protein-1; MCP-1), CC chemokine ligand 3 (CCL3; also known as macrophage inflammatory protein 1alpha; MIP-1α), CC chemokine ligand 4 (CCL4; also known as macrophage inflammatory protein 1beta; MIP-1β), CC chemokine ligand 5 (CCL5; also known as RANTES), CC chemokine ligand 6 (CCL6), CC chemokine ligand 7 (CCL7; also known as monocyte chemotactic protein-3; MCP-3), CC chemokine ligand 8 (CCL8; also known as monocyte chemotactic protein-2; MCP-2), CC chemokine ligand 9 (CCL9; also know as macrophage inflammatory protein-1gamma; MIP-1γ, or CCL10), CC chemokine ligand 11 (CCL11; also known as eotaxin-1), CC chemokine ligand 12 (CCL12; also known as monocyte chemotactic protein-5; MCP-5), CC chemokine ligand 13 (CCL13), CC chemokine ligand 14 (CCL14; also known as HCC-1), CC chemokine ligand 15 (CCL15; also known as macrophage inflammatory protein-5; MIP-5, or HCC-2), CC chemokine ligand 16 (CCL16; also known as liver-expressed chemokine; LEC, or Monotactin-1; MTN-1), CC chemokine ligand 17 (CCL17; also known as thymus and activation regulated chemokine; TARC), CC chemokine ligand 18 (CCL18; also known as pulmonary and activation-regulated chemokine; PARC), CC chemokine ligand 19 (CCL19; also known as macrophage inflammatory protein-3beta; MIP-3β, or EB/1 ligand chemokine; ELC), CC chemokine ligand 20 (CCL20; also known as macrophage inflammatory protein-3alpha; MIP-3α, or liver activation-regulated chemokine; LARC), CC chemokine ligand 21 (CCL21; also known as 6Ckine, or exodus-2, or secondary lymphoid-tissue chemokine; SLC), CC chemokine ligand 22 (CCL22), CC chemokine ligand 23 (CCL23; also known as macrophage inflammatory protein-3; MIP-3, or myeloid progenitor inhibitory factor-1; MPIF-1), CC chemokine ligand 24 (CCL24; also known as myeloid progenitor inhibitory factor-2; MPIF-2, or Eotaxin-2), CC chemokine ligand 25 (CCL25; also known as thymus expressed chemokine; TECK), CC chemokine ligand 26 (CCL26; also known as macrophage inflammatory protein-4alpha; MIP-4α, or Eotaxin-3, or IMAC), CC chemokine ligand 27 (CCL27; also known as IL-11 R-alpha-locus chemokine; ILC, or Skinkine, or Eskine, or cutaneous T-cell-attracting chemokine; CTACK) and CC chemokine ligand 28 (CCL28; also known as mucosae-associated epithelial chemokine; MEC, or CCK1, or SCYA28), Maraviroc (CCR5), Aplaviroc (CCR5), Vicriviroc (CCR5).

Known CXC chemokine receptor-related compounds include: the CXC chemokine ligand 1 (CXCL1; also known as neutrophil-activating protein-3; NAP-3, or melanoma growth stimulating activity alpha; MSGA-α), CXC chemokine ligand 2 (CXCL2; also known as macrophage inflammatory protein-2alpha; MIP-2α, or growth-regulated protein beta; Gro-beta), CXC chemokine ligand 3 (CXCL3; also known as macrophage inflammatory protein-2beta; MIP-2β), CXC chemokine ligand 4 (CXCL4; also known as platelet factor-4; PF4), CXC chemokine ligand 5 (CXCL5; also known as epithelial-derived neutrophil-activating peptide 78; ENA-78), CXC chemokine ligand 6 (CXCL6; also known as granulocyte chemotactic protein 2; GCP-2), CXC chemokine ligand 7 (CXCL7; also known as Pro-platelet basic protein; PPBP), CXC chemokine ligand 8 (CXCL8; also known as interleukin-8; IL-8), CXC chemokine ligand 9 (CXCL9; also known as monokine induced by gamma interferon; MIG), CXC chemokine ligand 10 (CXCL10; also known as 10 kDa interferon-gamma-induced protein; γ-IP10 or IP-10), CXC chemokine ligand 11 (CXCL11; also known as interferon-inducible T-cell alpha chemoattractant; I-TAC, or interferon-gamma-inducible protein 9; IP-9), CXC chemokine ligand 12 (CXCL12; also known as stromal-cell derived factor-1; SDF-1), CXC chemokine ligand 13 (CXCL13; also known as B lymphocyte chemoattractant; BLC), CXC chemokine ligand 14 (CXCL14; breast and kidney-expressed chemokine; BRAK), CXC chemokine ligand 15 (CXCL15; also known as lungkine), CXC chemokine ligand 16 (CXCL16), CXC chemokine ligand 17 (CXCL17; also known as VEGF co-regulated chemokine 1; VCC-1, or dendritic cell- and monocyte-attracting chemokine-like protein; DMC). The CX3 chemokine ligand 1 (CX3CL1; also known as Fractalkine or neurotactin). The XC chemokine ligand 1 (XCL1; also known as lymphotactin), and the XC chemokine ligand 2 (XCL2).

Known antagonists of chemokine receptors include; Repertaxin (CXCR2), TAK-779 (CCR5), TAK-220 (CCR5), TAK-652 (CCR5), AK692 (CCR5), CMPD167 (CCR5), BX-471 (CCR1), AMD3100 (CXCR4), AMD11070 (CXCR4), FC131 (CXCR4), MLN3897 (CCR1), CP-481715 (CCR1), GW-873140 (CCR5), propagermanium (GE-132) (CCR2), Resveratrol (CCR2), BMS CCR222 (CCR2), RS 102895 (CCR2), RS 504393 (CCR2), SB 225002 (CXCR2) and SB 265610 (CXCR2).

Angiotensin-related ailments include aliments that are related to increased or decreased production of angiotensin, and/or increased or decreased responsiveness of cells to angiotensin. Listed below are a number of conditions that have either been proposed to stem from a dysregulated angiotensin system, or, could potentially be treated using angiotensin-based interventions:
Chronic heart failure;
Atherosclerosis/ischemia;
Hypertension;
Hyperkalemia;
Preeclampsia;
Diabetes mellitus;
Diabetic retinopathy;
Sarcoidosis;
Alzheimer's Disease

However, it should be understood that the phrase angiotensin-related ailment is not limited thereto.

Known angiotensin receptor-related compounds include angiotensin II (AngII) and angiotensin III (AngIII). Known antagonists for ATR include: CGP-42112A (AT₂R antagonist; Sigma #C-160), Eprosartan (AT₁R; market name Teveten®, Abbott Laboratories USA), Losartan (AT₁R; market name Cozaar®, Merk & Co), Valsartan (AT₁R; market name Diovan®, Novartis), Telmisartan (AT₁R, market name Micardis®, Boehringer Ingelheim), Irbesartan (AT₁R, market name Avapro®, SanofiAventis), Olemsartan (AT₁R, market name Benicar®, Daiichi Sankyo Inc), PD123319 (AT₂R, Tocris), ZD-7115 (AT₁R), Saralasin ((Sar¹-Ala⁸)AngII), Sarthran ((Sar¹-Thr⁸)AngII) and DuP753 (AT₁R).

The discovery of the novel hetero-dimers of the present invention provides a context for certain experimental observations.

The ability of AngII to induce chemokine expression in glomerular endothelial cells of the rat was investigated with respect to renal disease pathogenesis (Wolf et al. (1997) Angiotensin II stimulates expression of the chemokine RANTES in rat glomerular endothelial cells. Role of the angiotensin type 2 receptor. J Clin Invest 100:1047-1058). AngII treatment led to an increase in mRNA and protein expression of the chemokine RANTES that was accompanied by an increase in monocyte recruitment. These effects could be abrogated if an AT₂R-specific antagonist was administered in conjunction with AngII, suggesting this receptor subtype mediated the observations reported. Similarly, AngIII treatment of renal cells and monocytes produced an increase in MCP-1 mRNA and protein expression, with supernatants from cultured cells also producing a chemotactic response by macrophages (Ruiz-Ortega, M. (2000) Angiotensin III increases MCP-1 and activates NF-kappaB and AP-1 in cultured mesangial and mononuclear cells. Kidney International 57:2285-2298). Furthermore, AngIII activated the transcription factor NF-κB, which was required for the elevated expression of MCP-1. Following 3 days of AngII treatment, rats exhibited increased renal expression of TNFα and MCP-1 that again correlated with increased NF-κB activity and macrophage recruitment to renal tissue (Ruiz-Ortega, M. (2002) Angiotensin II regulates the synthesis of proinflammatory cytokines and chemokines in the kidney. Kidney International Supplementary. 12-22).

Finally, the contribution of CCR2b to the development of renal injury was assessed in a salt-sensitive, AngII model of hypertension (Elmarakby, A. (2007) Chemokine Receptor 2b Inhibition Provides Renal Protection in Angiotensin II Salt Hypertension. Hypertension 50:1069-1076). Hypersensitive animals administered AngII and a high salt diet demonstrated a comparable BP to animals also treated with a CCR2b antagonist (RS102895), but had significantly higher levels of NF-κB and TNFα expression that was associated with increased macrophage infiltration. The authors concluded that the CCR2b antagonist protects the kidney in hypertension by reducing the inflammatory response.

Regarding the pathogenesis of hypertension and atherosclerosis, researchers utilised a mouse strain lacking the CCR2 receptor (CCR2^{-/-}) to assess the role of this receptor in mediating macrophage infiltration (Bush, E. (2000) CC chemokine receptor 2 is required for macrophage infiltration and vascular hypertrophy in angiotensin II-induced hypertension. Hypertension 36:360-363). Following 7 days of AngII treatment to induce hypertension, CCR2^{-/-} mice were found to have significantly less macrophage recruitment and reduced vascular hypertrophy compared with WT animals. An apolipoprotein E-deficient mouse model, which mirrors the atherosclerotic phenotype in humans, was used to determine the mechanism(s) responsible for the beneficial actions of AT₁R antagonists in the treatment of atherosclerosis (Dol, F. (2001) Angiotensin AT₁ receptor antagonist irbesartan decreases lesion size, chemokine expression, and macrophage accumulation in apolipoprotein E-deficient mice. Journal Cardiovascular Pharmacology 38:395-405). Animals treated with antagonist showed a reduced lesion size after 12 weeks and in addition, had significantly lower levels of MCP-1 and MIP-1α mRNA expression.

The effect of an ACE-inhibitor Ramiprilat upon chemokine release, NF-κB activity and AT₁R expression was measured in AngII treated monocytes (Schmeisser, A. (2004) ACE inhibition lowers angiotensin II-induced chemokine expression by reduction of NF-kappaB activity and AT1 receptor expression. Biochem Biophys Res Commun 325:532-540). AngII-mediated upregulation of MIP-1 and interleukin-8 chemokines was inhibited by an AT1R-specific antagonist (Losartan) but not an AT₂R antagonist, while Ramiprilat also suppressed AngII-induced upregulation of MIP-1, which correlated with a down-regulation of NF-κB activity and a concurrent decrease in AT₁R expression. Furthermore, the ability of CXCR2 antagonists to inhibit macrophage infiltration in response to AngII has also been shown in the context of atherosclerosis (Abu Nabah, Y. (2007) CXCR2 blockade impairs angiotensin II-induced CC chemokine synthesis and mononuclear leukocyte infiltration. Arterioscler Thromb Vasc Biol 27:2370-2376). Intraperitoneal AngII administration caused an increase in CC and CXC chemokine levels that preceded the infiltration of leukocytes, and was abrogated in the presence of a CXCR2 antagonist. Leukocyte infiltration was also inhibited by Losartan, indicating the AngII effects were mediated by AT₁R.

In one embodiment, the present invention provides a method for the treatment of a patient suffering from an angiotensin-related ailment by administering a therapeutically effective amount of a chemokine-related compound selected from, but not limited to, the group: CC chemokine ligand 1 (CCL1), CC chemokine ligand 2 (CCL2; also known as monocyte chemotactic protein-1; MCP-1), CC chemokine ligand 3 (CCL3; also known as macrophage inflammatory protein 1alpha; MIP-1α), CC chemokine ligand 4 (CCL4; also known as macrophage inflammatory protein 1beta; MIP-1β), CC chemokine ligand 5 (CCL5; also known as RANTES), CC chemokine ligand 6 (CCL6), CC chemokine ligand 7 (CCL7; also known as monocyte chemotactic protein-3; MCP-3), CC chemokine ligand 8 (CCL8; also known as monocyte chemotactic protein-2; MCP-2), CC chemokine ligand 9 (CCL9; also know as macrophage inflammatory protein-1gamma; MIP-1γ, or CCL10), CC chemokine ligand 11 (CCL11; also known as eotaxin-1), CC chemokine ligand 12 (CCL12; also known as monocyte chemotactic protein-5; MCP-5), CC chemokine ligand 13 (CCL13), CC chemokine ligand 14 (CCL14; also known as HCC-1), CC chemokine ligand 15 (CCL15; also known as macrophage inflammatory protein-5; MIP-5, or HCC-2), CC chemokine ligand 16 (CCL16; also known as liver-expressed chemokine; LEC, or Monotactin-1; MTN-1), CC chemokine ligand 17 (CCL17; also known as thymus and activation regulated chemokine; TARC), CC chemokine ligand 18 (CCL18; also known as pulmonary and activation-regulated chemokine; PARC), CC chemokine ligand 19 (CCL19; also known as macrophage inflammatory protein-3beta; MIP-3β, or EB/1 ligand chemokine; ELC), CC chemokine ligand 20 (CCL20; also known as macrophage inflammatory protein-3alpha; MIP-3α, or liver activation-regulated chemokine; LARC), CC chemokine ligand 21 (CCL21; also known as 6Ckine, or exodus-2, or secondary lymphoid-tissue chemokine; SLC), CC chemokine ligand 22 (CCL22), CC chemokine ligand 23 (CCL23; also known as macrophage inflammatory protein-3; MIP-3, or myeloid progenitor inhibitory factor-1; MPIF-1), CC chemokine ligand 24 (CCL24; also known as myeloid progenitor inhibitory factor-2; MPIF-2, or Eotaxin-2), CC chemokine ligand 25 (CCL25; also known as thymus expressed chemokine; TECK), CC chemokine ligand 26 (CCL26; also known as macrophage inflammatory protein-4alpha; MIP-4α, or Eotaxin-3, or IMAC), CC chemokine ligand 27 (CCL27; also known as IL-11 R-alpha-locus chemokine; ILC, or Skinkine, or Eskine, or cutaneous T-cell-attracting chemokine; CTACK), CC chemokine ligand 28 (CCL28; also known as mucosae-associated epithelial chemokine; MEC, or CCK1, or SCYA28), the CXC chemokine ligand 1 (CXCL1; also known as neutrophil-activating protein-3; NAP-3, or melanoma growth stimulating activity alpha; MSGA-α), CXC chemokine ligand 2 (CXCL2; also known as macrophage inflammatory protein-2alpha; MIP-2α, or growth-regulated protein beta; Gro-beta), CXC chemokine ligand 3 (CXCL3; also known as macrophage inflammatory protein-2beta; MIP-2β), CXC chemokine ligand 4 (CXCL4; also known as platelet factor-4; PF4), CXC chemokine ligand 5 (CXCL5; also known as epithelial-derived neutrophil-activating peptide 78; ENA-78), CXC chemokine ligand 6 (CXCL6; also known as granulocyte chemotactic protein 2; GCP-2), CXC chemokine ligand 7 (CXCL7; also known as Pro-platelet basic protein; PPBP), CXC chemokine ligand 8 (CXCL8; also known as interleukin-8; IL-8), CXC chemokine ligand 9 (CXCL9; also known as monokine induced by gamma interferon; MIG), CXC chemokine ligand 10 (CXCL10; also known as 10 kDa interferon-gamma-induced protein; γ-IP10 or IP-10), CXC chemokine ligand 11 (CXCL11; also known as interferon-inducible T-cell alpha chemoattractant; I-TAC, or interferon-gamma-inducible protein 9; IP-9), CXC chemokine ligand 12 (CXCL12; also known as stromal-cell derived factor-1; SDF-1), CXC chemokine ligand 13 (CXCL13; also known as B lymphocyte chemoattractant; BLC), CXC chemokine ligand 14 (CXCL14; breast and kidney-expressed chemokine; BRAK), CXC chemokine ligand 15 (CXCL15; also known as lungkine), CXC chemokine ligand 16 (CXCL16), CXC chemokine ligand 17 (CXCL17; also known as VEGF co-regulated chemokine 1; VCC-1, or dendritic cell- and monocyte-attracting chemokine-like protein; DMC), CX3 chemokine ligand 1 (CX3CL1; also known as Fractalkine or neurotactin), XC chemokine ligand 1 (XCL1; also known as lymphotactin), XC chemokine ligand 2 (XCL2), Repertaxin (CXCR2), Maraviroc (CCR5), Aplaviroc (CCR5), Vicriviroc (CCR5), TAK-779 (CCR5), TAK-220 (CCR5), TAK-652 (CCR5), AK692 (CCR5), CMPD167 (CCR5), BX-471 (CCR1), AMD3100 (CXCR4), AMD11070 (CXCR4), FC131 (CXCR4), MLN3897 (CCR1), CP-481715 (CCR1) and GW-873140 (CCR5), propagermanium (GE-132) (CCR2), Resveratrol (CCR2), BMS CCR222 (CCR2), RS 102895 (CCR2), RS 504393 (CCR2), SB 225002 (CXCR2) and SB 265610 (CXCR2).

In one embodiment, the present invention provides a method for the treatment of a patient suffering from a chemokine-related ailment by administering a therapeutically effective amount of an angiotensin-related compound selected from, but not limited to, the group: angiotensin II (AngII), angiotensin III (AngIII), CGP-42112A (AT₂R; Sigma #C-160), Eprosartan (AT₁R; market name Teveten®, Abbott Laboratories USA), Losartan (AT₁R; market name Cozaar®, Merk & Co), Valsartan (AT₁R; market name Diovan®, Novartis), Telmisartan (AT₁R, market name Micardis®, Boehringer Ingelheim), Irbesartan (AT₁R, market name Avapro®, SanofiAventis), Olemsartan (AT₁R, market name Benicar®, Daiichi Sankyo Inc), Candesartan (market name Atacand®, AstraZeneca) PD123319 (AT₂R, Tocris), ZD-7115 (AT₁R), Saralasin ((Sar¹-Ala⁸)AngII), Sarthran ((Sar¹-Thr⁸)AngII) and DuP753 (AT₁R).

The present invention also includes a method for screening a test compound for potential therapeutic activity against an angiotensin-related ailment using a detector capable of detecting changes in receptor activity, the method comprising the steps of:
a) exposing the test compound to a chemokine receptor;
b) assessing whether and/or the extent to which the activity of the chemokine receptor is modulated compared to activity of the chemokine receptor in the absence of the test compound;
said modulation being indicative of potential therapeutic activity against the angiotensin-related ailment.

The present invention also includes a method for screening a test compound for potential therapeutic activity against an angiotensin-related ailment, the method comprising the steps of:
a) exposing the test compound to a chemokine receptor;
b) assessing the extent to which the activity of the chemokine receptor is modulated;
said modulation being indicative of potential therapeutic activity against the angiotensin-related ailment.

In one embodiment, the method for screening a test compound for potential therapeutic activity against an angiotensin-related ailment using a detector capable of detecting changes in receptor activity comprises the step of:
administering said test compound to an animal.

In one embodiment, the method for screening a test compound for potential therapeutic activity against an angiotensin-related ailment using a detector capable of detecting changes in receptor activity comprises the steps of:
contacting a cell expressing chemokine receptor with said test compound;
detecting a change in chemokine receptor activity.

Methods for assessing the extent to which the activity of a chemokine receptor is modulated and detectors capable of detecting changes in receptor activity are well-known to those skilled in the art.

The present invention also includes a method for screening a test compound for potential therapeutic activity against a chemokine-related ailment using a detector capable of detecting changes in receptor activity, the method comprising the steps of:
a) exposing the test compound to an angiotensin receptor;
b) assessing whether and/or the extent to which the activity of the angiotensin receptor is modulated compared to activity of the angiotensin receptor in the absence of the test compound;
said modulation being indicative of potential therapeutic activity against the chemokine-related ailment.

The present invention also includes a method for screening a test compound for potential therapeutic activity against a chemokine-related ailment, the method comprising the steps of:
a) exposing the test compound to an angiotensin receptor;
b) assessing the extent to which the activity of the angiotensin receptor is modulated;
said modulation being indicative of potential therapeutic activity against the chemokine-related ailment.

In one embodiment, the method for screening a test compound for potential therapeutic activity against a chemokine-related ailment using a detector capable of detecting changes in receptor activity comprises the step of:
administering said test compound to an animal.

In one embodiment, the method for screening a test compound for potential therapeutic activity against a chemokine-related ailment using a detector capable of detecting changes in receptor activity comprises the steps of:
contacting a cell expressing angiotensin receptor with said test compound;
detecting a change in angiotensin receptor activity.

Methods for assessing the extent to which the activity of an angiotensin receptor is modulated and detectors capable of detecting changes in receptor activity are well-known to those skilled in the art.

The present invention comprises a method for screening a test compound for chemokine receptor/angiotensin receptor hetero-dimer/-oligomer selective activity using a detector capable of detecting changes in receptor activity, the method comprising the steps of:
a) determining whether, and/or the extent to which, the test compound interacts with the angiotensin receptor while the angiotensin receptor is associated with the chemokine receptor; and
b) determining whether, and/or the extent to which the test compound interacts with the angiotensin receptor in the absence of the chemokine receptor;
   such that a test compound that exhibits greater affinity and/or potency and/or efficacy when interacting with the angiotensin receptor while the angiotensin receptor is associated with the chemokine receptor is selective for the chemokine receptor/angiotensin receptor hetero-dimer/-oligomer.

The present invention comprises a method for screening a test compound for chemokine receptor/angiotensin receptor hetero-dimer/-oligomer selective activity, the method comprising the steps of:
a) determining whether, and/or the extent to which, the test compound interacts with the angiotensin receptor while the angiotensin receptor is associated with the chemokine receptor; and
b) if the test compound interacts with the angiotensin receptor while the angiotensin receptor is associated with the chemokine receptor, determining whether, or the extent to which the test compound interacts with the angiotensin receptor in the absence of the chemokine receptor;
   such that a test compound that exhibits greater affinity and/or potency and/or efficacy when interacting with the angiotensin receptor while the angiotensin receptor is associated with the chemokine receptor is selective for the chemokine receptor/angiotensin receptor hetero-dimer/-oligomer.

In one embodiment, the method for screening a test compound for chemokine receptor/angiotensin receptor hetero-dimer/-oligomer selective activity using a detector capable of detecting changes in receptor activity comprises the step of:
administering said test compound to an animal.

In one embodiment, the method for screening a test compound for chemokine receptor/angiotensin receptor hetero-dimer/-oligomer selective activity using a detector capable of detecting changes in receptor activity comprises the steps of:
contacting a cell expressing angiotensin receptor and chemokine receptor with said test compound;
detecting a change in angiotensin receptor or chemokine receptor activity.

The present invention comprises a method for screening a test compound for chemokine receptor/angiotensin receptor hetero-dimer/-oligomer selective activity, the method comprising the steps of:
a) determining whether, and/or the extent to which, the test compound interacts with the chemokine receptor while the chemokine receptor is associated with the angiotensin receptor; and
b) if the test compound interacts with the chemokine receptor while the chemokine receptor is associated with the angiotensin receptor, determining whether, or the extent to which the test compound interacts with the chemokine receptor in the absence of the angiotensin receptor;
   such that a test compound that exhibits greater affinity and/or potency and/or efficacy when interacting with the chemokine receptor while the chemokine receptor is associated with the angiotensin receptor is selective for the chemokine receptor/angiotensin receptor hetero-dimer/-oligomer.

The present invention further provides a method for screening a test compound for chemokine receptor/angiotensin receptor hetero-dimer/-oligomer selective activity using a detector capable of detecting changes in receptor activity, the method comprising the step of:
determining whether, and/or the extent to which, the test compound interacts with the chemokine receptor while the chemokine receptor is associated with the angiotensin receptor compared to whether, and/or the extent to which the test compound interacts with the chemokine receptor in the absence of the angiotensin receptor;
   such that a test compound that exhibits greater affinity and/or potency and/or efficacy when interacting with the chemokine receptor while the chemokine receptor is associated with the angiotensin receptor is selective for the chemokine receptor/angiotensin receptor hetero-dimer/-oligomer.

In one embodiment, the method for screening a test compound for chemokine receptor/angiotensin receptor hetero-dimer/-oligomer selective activity using a detector capable of detecting changes in receptor activity comprises the step of:
administering said test compound to an animal.

In one embodiment, the method for screening a test compound for chemokine receptor/angiotensin receptor hetero-dimer/-oligomer selective activity using a detector capable of detecting changes in receptor activity comprises the steps of:
contacting a cell expressing angiotensin receptor and chemokine receptor with said test compound;
detecting a change in angiotensin receptor or chemokine receptor activity.

In one aspect of the invention, there is provided a method for screening a test compound for chemokine receptor/angiotensin receptor hetero-dimer/-oligomer selective antagonism or partial agonism, the method comprising the steps of:
a) determining whether, and/or the extent to which, the test compound is an antagonist or partial agonist of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising the chemokine receptor;
   iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting a decrease in the signal as a determination of whether and/or the extent to which the test compound is an antagonist or partial agonist of the chemokine receptor/angiotensin receptor hetero-dimer/-oligomer;
c) if the test compound is an antagonist or partial agonist of the chemokine receptor/angiotensin receptor hetero-dimer/-oligomer, determining whether, or the extent to which the test compound is an antagonist or partial agonist of the chemokine receptor in the absence of the angiotensin receptor and the angiotensin receptor in the absence of the chemokine receptor; such that a test compound that exhibits greater antagonistic or partial agonistic properties when interacting with the chemokine receptor/angiotensin receptor hetero-dimer/-oligomer is selective for the chemokine receptor/angiotensin receptor hetero-dimer/-oligomer.

In one aspect of the invention, there is provided a method for screening a test compound for chemokine receptor/angiotensin receptor hetero-dimer/-oligomer selective antagonism or selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising the chemokine receptor;
   iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting a decrease in the signal as a determination of whether and/or the extent to which the test compound is an antagonist, partial agonist or negative allosteric modulator of the chemokine receptor/angiotensin receptor hetero-dimer/-oligomer and optionally;
c) determining whether, or the extent to which the test compound is an antagonist, partial agonist, or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

In one embodiment, the method for screening a test compound for chemokine receptor/angiotensin receptor hetero-dimer/-oligomer selective antagonism, selective partial agonism or selective negative allosteric modulation using a detector capable of detecting changes in receptor activity comprises the step of:
administering said test compound to an animal.

In one embodiment, the method for screening a test compound for chemokine receptor/angiotensin receptor hetero-dimer/-oligomer selective antagonism, selective partial agonism or selective negative allosteric modulation using a detector capable of detecting changes in receptor activity comprises the steps of:
contacting a cell expressing angiotensin receptor and chemokine receptor with said test compound;
detecting a change in angiotensin receptor or chemokine receptor activity.

In one aspect of the invention, there is provided a method for screening a test compound for chemokine receptor/angiotensin receptor hetero-dimer/-oligomer selective antagonism or partial agonism, the method comprising the steps of:
a) determining whether, and/or the extent to which, the test compound is an antagonist or partial agonist of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising the angiotensin receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the angiotensin receptor;
b). detecting a decrease in the signal as a determination of whether and/or the extent to which the test compound is an antagonist or partial agonist of the chemokine receptor/angiotensin receptor hetero-dimer/-oligomer;
c) if the test compound is an antagonist or partial agonist of the chemokine receptor/angiotensin receptor hetero-dimer/-oligomer, determining whether, or the extent to which the test compound is an antagonist or partial agonist of the chemokine receptor in the absence of the angiotensin receptor and the angiotensin receptor in the absence of the chemokine receptor; such that a test compound that exhibits greater antagonistic or partial agonistic properties when interacting with the chemokine receptor/angiotensin receptor hetero-dimer/-oligomer is selective for the chemokine receptor/angiotensin receptor hetero-dimer/-oligomer.

In one aspect of the invention, there is provided a method for screening a test compound for chemokine receptor/angiotensin receptor hetero-dimer/-oligomer selective antagonism, selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising the angiotensin receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the angiotensin receptor;
b). detecting a decrease in the signal as a determination of whether and/or the extent to which the test compound is an antagonist, partial agonist or negative allosteric modulator of the chemokine receptor/angiotensin receptor hetero-dimer/-oligomer and optionally;
c) determining whether, or the extent to which the test compound is an antagonist, partial agonist or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

In one embodiment, the method for screening a test compound for chemokine receptor/angiotensin receptor hetero-dimer/-oligomer selective antagonism, selective partial agonism or selective negative allosteric modulation using a detector capable of detecting changes in receptor activity comprises the step of:
administering said test compound to an animal.

In one embodiment, the method for screening a test compound for chemokine receptor/angiotensin receptor hetero-dimer/-oligomer selective antagonism, selective partial agonism or selective negative allosteric modulation using a detector capable of detecting changes in receptor activity comprises the steps of:
contacting a cell expressing angiotensin receptor and chemokine receptor with said test compound;
detecting a change in angiotensin receptor or chemokine receptor activity.

In one aspect of the invention, there is provided a method for screening a test compound for chemokine receptor/angiotensin receptor hetero-dimer/-oligomer selective inverse agonism, the method comprising the steps of:
b) determining whether, and/or the extent to which, the test compound is an inverse agonist of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising a constitutively active chemokine receptor;
   wherein proximity of the first and second reporter components generates a signal; and wherein the modulator modulates the association of the interacting group with the chemokine receptor;
b) detecting a decrease in the signal as a determination of whether and/or the extent to which the test compound is an inverse agonist of the chemokine receptor/angiotensin receptor hetero-dimer/-oligomer;
c) if the test compound is an inverse agonist of the chemokine receptor/angiotensin receptor hetero-dimer/-oligomer, determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine/angiotensin receptor hetero-dimer/-oligomer.

In one aspect of the invention, there is provided a method for screening a test compound for chemokine receptor/angiotensin receptor hetero-dimer/-oligomer selective inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising a constitutively active chemokine receptor;
   wherein proximity of the first and second reporter components generates a signal; and wherein the modulator modulates the association of the interacting group with the chemokine receptor;
b) detecting a decrease in the signal as a determination of whether and/or the extent to which the test compound is an inverse agonist of the chemokine receptor/angiotensin receptor hetero-dimer/-oligomer and optionally;
c) determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine/angiotensin receptor hetero-dimer/-oligomer.

In one embodiment, the method for screening a test compound for chemokine receptor/angiotensin receptor hetero-dimer/-oligomer selective inverse agonism using a detector capable of detecting changes in receptor activity comprises the step of:
administering said test compound to an animal.

In one embodiment, the method for screening a test compound for chemokine receptor/angiotensin receptor hetero-dimer/-oligomer selective inverse agonism using a detector capable of detecting changes in receptor activity comprises the steps of:
contacting a cell expressing angiotensin receptor and chemokine receptor with said test compound;
detecting a change in angiotensin receptor or chemokine receptor activity.

In one aspect of the invention, there is provided a method for screening a test compound for chemokine receptor/angiotensin receptor hetero-dimer/-oligomer inverse agonism, the method comprising the steps of:
a) determining whether, and/or the extent to which, the test compound is an inverse agonist of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising a constitutively active angiotensin receptor;
   wherein proximity of the first and second reporter components generates a signal; and wherein the modulator modulates the association of the interacting group with the angiotensin receptor;
b) detecting a decrease in the signal as a determination of whether and/or the extent to which the test compound is an inverse agonist of the chemokine receptor/angiotensin receptor hetero-dimer/-oligomer;
c) if the test compound is an inverse agonist of the chemokine receptor/angiotensin receptor hetero-dimer/-oligomer, determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine receptor/angiotensin receptor hetero-dimer/-oligomer.

In one aspect of the invention, there is provided a method for screening a test compound for chemokine receptor/angiotensin receptor hetero-dimer/-oligomer inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising a constitutively active angiotensin receptor;
   wherein proximity of the first and second reporter components generates a signal; and wherein the modulator modulates the association of the interacting group with the angiotensin receptor;
b) detecting a decrease in the signal as a determination of whether and/or the extent to which the test compound is an inverse agonist of the chemokine receptor/angiotensin receptor hetero-dimer/-oligomer and optionally;
c) determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine receptor/angiotensin receptor hetero-dimer/-oligomer.

In one embodiment, the method for screening a test compound for chemokine receptor/angiotensin receptor hetero-dimer/-oligomer selective inverse agonism using a detector capable of detecting changes in receptor activity comprises the step of:
administering said test compound to an animal.

In one embodiment, the method for screening a test compound for chemokine receptor/angiotensin receptor hetero-dimer/-oligomer selective inverse agonism using a detector capable of detecting changes in receptor activity comprises the steps of:
contacting a cell expressing angiotensin receptor and chemokine receptor with said test compound;
detecting a change in angiotensin receptor or chemokine receptor activity.

The present invention further provides a method for screening a test compound for chemokine receptor/angiotensin receptor hetero-dimer/-oligomer selective positive allosteric modulation using a detector capable of detecting changes in receptor activity, the method comprising the steps of:
a) determining whether and/or the extent to which the test compound is a positive allosteric modulator of the chemokine receptor/angiotensin receptor hetero-dimer/-oligomer, by contacting said test compound with a system comprising:
   i). a first agent, comprising the angiotensin receptor coupled to a first reporter component;
   ii). a second agent, comprising an interacting group coupled to a second reporter component;
   iii). a third agent, comprising the chemokine receptor;
   iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting an increase in the signal as a determination of whether and/or the extent to which the test compound is a positive allosteric modulator of the chemokine receptor/angiotensin receptor hetero-dimer/-oligomer and optionally;
c) determining whether and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine/angiotensin receptor hetero-dimer/-oligomer.

In one embodiment, the method for screening a test compound for chemokine receptor/angiotensin receptor hetero-dimer/-oligomer selective positive allosteric modulation using a detector capable of detecting changes in receptor activity comprises the step of:
administering said test compound to an animal.

In one embodiment, the method for screening a test compound for chemokine receptor/angiotensin receptor hetero-dimer/-oligomer selective positive allosteric modulation using a detector capable of detecting changes in receptor activity comprises the steps of:
contacting a cell expressing angiotensin receptor and chemokine receptor with said test compound;
detecting a change in angiotensin receptor or chemokine receptor activity.

The present invention further provides a method for screening a test compound for chemokine receptor/angiotensin receptor hetero-dimer/-oligomer selective positive allosteric modulation using a detector capable of detecting changes in receptor activity, the method comprising the steps of:
a) determining whether and/or the extent to which the test compound is a positive allosteric modulator of the chemokine receptor/angiotensin receptor hetero-dimer/-oligomer, by contacting said test compound with a system comprising:
   i). a first agent, comprising the chemokine receptor coupled to a first reporter component;
   ii). a second agent, comprising an interacting group coupled to a second reporter component;
   iii). a third agent, comprising the angiotensin receptor;
   iv). an agonist of the angiotensin receptor, the chemokine receptor and/or the chemokine receptor/angiotensin receptor hetero-dimer/-oligomer;
   wherein proximity of the first and second reporter components generates a signal; and wherein the modulator modulates the association of the interacting group with the angiotensin receptor;
b) detecting an increase in the signal as a determination of whether and/or the extent to which the test compound is a positive allosteric modulator of the chemokine receptor/angiotensin receptor hetero-dimer/-oligomer and optionally;
c) determining whether and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine receptor/angiotensin receptor hetero-dimer/-oligomer.

In one embodiment, the method for screening a test compound for chemokine receptor/angiotensin receptor hetero-dimer/-oligomer selective positive allosteric modulation using a detector capable of detecting changes in receptor activity comprises the step of:
administering said test compound to an animal.

In one embodiment, the method for screening a test compound for chemokine receptor/angiotensin receptor hetero-dimer/-oligomer selective positive allosteric modulation using a detector capable of detecting changes in receptor activity comprises the steps of:
contacting a cell expressing angiotensin receptor and chemokine receptor with said test compound;
detecting a change in angiotensin receptor or chemokine receptor activity.

In a preferred embodiment of the invention, the step of determining whether, and/or the extent to which, the test compound interacts with the chemokine receptor while the chemokine receptor is associated with the angiotensin receptor; and/or the step of determining whether, and/or the extent to which, the test compound interacts with the angiotensin receptor while the angiotensin receptor is associated with the chemokine receptor are performed by way of the methods described in the applicant's co-pending international patent application “Detection System and Uses Therefor”, PCT/AU2007/001722 (published as WO 2008/055313).

However, for the sake of clarity, it should be understood that the methods of the present invention are not restricted to methods where the step of determining whether, and/or the extent to which, the test compound interacts with the angiotensin receptor while the angiotensin receptor is associated with the chemokine receptor; and/or the step of determining whether, and/or the extent to which, the test compound interacts with the chemokine receptor while the chemokine receptor is associated with the angiotensin receptor are performed by way of the methods described in the applicant's co-pending international patent application “Detection System and Uses Therefor”, PCT/AU2007/001722 (published as WO 2008/055313).

Alternate methods of determining whether, and/or the extent to which, the test compound interacts with the angiotensin receptor while the angiotensin receptor is associated with the chemokine receptor; and/or the step of determining whether, and/or the extent to which, the test compound interacts with the chemokine receptor while the chemokine receptor is associated with the angiotensin receptor include assays observing a change in coupling to signalling pathways such as a change in G-protein utilisation, ligand binding assays, signalling assays such as those monitoring changes in Ca²⁺, inositol phosphate, cyclic adenosine monophosphate (cAMP), extracellular-signal regulated kinase (ERK) and/or mitogen-activated protein kinase (MAPK), receptor trafficking assays, beta-arrestin translocation assays, enzyme-linked immunosorbent assays (ELISAs) and any other assay that can detect a change in receptor function as a result of receptor heterodimerization.

The present invention includes selective agonists and/or antagonists and/or inverse agonists of the chemokine receptor/angiotensin receptor hetero-dimer/-oligomer.

The present invention includes selective agonists and/or selective antagonists and/or selective inverse agonists and/or selective allosteric modulators of the chemokine receptor/angiotensin receptor hetero-dimer/-oligomer.

The present invention comprises a cell, or fraction of a cell, in which both a chemokine receptor and an angiotensin receptor are over-expressed.

The present invention comprises a cell, or fraction of a cell, in which a chemokine receptor is over-expressed with an endogenously expressed angiotensin receptor.

The present invention comprises a cell, or fraction of a cell, in which an angiotensin receptor is over-expressed with an endogenously expressed chemokine receptor.

Throughout this specification, unless the context requires otherwise, the phrase “fraction of a cell’ includes, without limitation, cell membrane preparations. As would be understood by a person skilled in the art, cell membrane preparations are useful in binding assays, or as antigens against which antibodies, including antibody therapeutics, may be raised.

The present invention comprises a cell in which both a chemokine receptor and an angiotensin receptor are over-expressed.

The present invention comprises a cell in which a chemokine receptor is over-expressed with an endogenously expressed angiotensin receptor.

The present invention comprises a cell in which an angiotensin receptor is over-expressed with an endogenously expressed chemokine receptor.

The phrase “over-expressed”, as used herein in the context of receptors, refers to an abnormal level of expression of the receptor within the cell relative to the natural level of expression. This may include a level of expression considered to be within the physiological range, but expressed in cells not normally expressing the receptor. This may also include a level of expression considered to be within the physiological range, but in cells not normally expressing the receptors modified in any way, such as by fusion to other proteins or by the addition of immunolabels. Cells in which a receptor is over-expressed may be identified by standard assay techniques well known in the art.

As used herein the term “patient” refers to any animal that may be suffering from one or more of angiotensin- or chemokine-related ailments. Most preferably the animal is a mammal. The term will be understood to include for example human, farm animals (i.e., cattle, horses, goats, sheep and pigs), household pets (i.e., cats and dogs) and the like.

The phrase “therapeutically effective amount” as used herein refers to an amount sufficient to modulate a biological activity associated with the interaction of angiotensin receptor agonist, inverse agonist, antagonist or allosteric modulator with the angiotensin receptor or chemokine receptor agonist, inverse agonist, antagonist or allosteric modulator with the chemokine receptor or of angiotensin receptor/chemokine receptor hetero-dimer/oligomer-specific agonist, inverse agonist, antagonist or allosteric modulator with an angiotensin receptor/chemokine receptor hetero-dimer/oligomer.

In the context of aspects of the invention where both a chemokine receptor-related compound, such as and without limitation a chemokine agonist, inverse agonist, antagonist or allosteric modulator and an angiotensin receptor-related compound, such as and without limitation, an angiotensin agonist, inverse agonist, antagonist or allosteric modulator are administered in combination, a therapeutically effective amount of a chemokine receptor-related compound, and a therapeutically effective amount of an angiotensin receptor-related compound in combination may be lower than therapeutically effective amounts of chemokine receptor-related compound when administered alone. That is, the administration of a chemokine receptor-related compound and a angiotensin receptor-related compound in combination may generate a therapeutic effect at what would otherwise be sub-therapeutic doses of either.

Medicaments of the invention, in various aspects, may be administered by injection, or prepared for oral, pulmonary, nasal or for any other form of administration. Preferably the medicaments are administered, for example, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is in one aspect at least suitable for the form in which the medicament has been prepared. The mode of administration for the most effective response is in one aspect determined empirically and the means of administration described below are given as examples, and do not limit the method of delivery of the composition of the present invention in any way. All the above formulations are commonly used in the pharmaceutical industry and are commonly known to suitably qualified practitioners.

The medicaments of the invention in certain aspects may include pharmaceutically acceptable nontoxic excipients and carriers and administered by any parenteral techniques such as subcutaneous, intravenous and intraperitoneal injections. In addition the formulations may optionally contain one or more adjuvants.

The pharmaceutical forms suitable for injectable use optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist, their transport across cell membrane. Alternatively or in addition such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminum monostearate and gelatin.

Sterile injectable solutions are prepared by incorporating the active compounds in the required amount in an appropriate solvent with various of the other ingredients enumerated above, as required, followed by filtered sterilisation. Generally, dispersions are prepared by incorporating the various sterilised active ingredient into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the active ingredient plus any additional desired ingredient from previously sterile-filtered solution thereof.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the compounds described as part of the invention (or a chemically modified form thereof), and inert ingredients which allow for protection against the stomach environment, and release of the biologically active material in the intestine.

For the chemokine receptor-related compounds or angiotensin receptor-related compounds of the invention the location of release may be the stomach, the small intestine (the duodenum, the jejunum, or the ileum), or the large intestine. One skilled in the art has available formulations that will not dissolve in the stomach, yet will release the material in the duodenum or elsewhere in the intestine. In one aspect, the release will avoid the deleterious effects of the stomach environment, either by protection of the composition or by release of the compounds beyond the stomach environment, such as in the intestine.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules consist of a hard shell (such as gelatin) for delivery of dry therapeutic i.e. powder; for liquid forms, a soft gelatin shell may be used. The shell material of cachets in certain aspects is thick starch or other edible paper. For pills, lozenges, moulded tablets or tablet triturates, moist massing techniques are also contemplated, without limitation.

In certain aspects, the therapeutic is included in the formulation as fine multiparticulates in the form of granules or pellets of particle size about 1 mm. The formulation of the material for capsule administration is, in certain aspects, a powder, lightly compressed plugs or even as tablets. In one aspect, the therapeutic could be prepared by compression.

Colourants and flavouring agents are optionally all be included. For example, compounds may be formulated (such as, and without limitation, by liposome or microsphere encapsulation) and then further contained within an edible product, such as a refrigerated beverage containing colorants and flavouring agents.

The volume of the therapeutic is, in one aspect, diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the therapeutic compounds together to form a hard tablet and include, without limitation, materials from natural products such as acacia, tragacanth, starch and gelatin. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

Antifrictional agents are optionally included in the formulation of the therapeutic to prevent sticking during the formulation process. Lubricants are optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants include sodium lauryl sulfate, magnesium lauryl sulfate, polyethylene glycol of various molecular weights, and Carbowax 4000 and 6000.

Glidants that improve the flow properties of the compound during formulation and to aid rearrangement during compression are optionally added. The glidants include without limitation starch, talc, pyrogenic silica and hydrated silicoaluminate.

To aid dissolution of the therapeutic into the aqueous environment, a surfactant is added in certain embodiments as a wetting agent. Surfactants include, for example and without limitation, anionic detergents such as sodium lauryl sulfate, dioctyl sodium sulfosuccinate and dioctyl sodium sulfonate. Cationic detergents are optionally used and include, without limitation, benzalkonium chloride or benzethomium chloride. The list of potential nonionic detergents that are contemplated in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants are present in the formulation of the compounds either alone or as a mixture in different ratios.

Additives that potentially enhance uptake of the compounds are for instance and without limitation the fatty acids oleic acid, linoleic acid and linolenic acid.

Controlled release formulations are also contemplated. In certain aspects, the compounds are incorporated into an inert matrix that permits release by either diffusion or leaching mechanisms i.e., gums. In some aspects, slowly degenerating matrices may also be incorporated into the formulation. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings have a delayed release effect.

In other aspects, a mix of materials is used to provide the optimum film coating. Film coating is carried out, for example and without limitation, in a pan coater or in a fluidized bed or by compression coating.

Also contemplated herein is pulmonary delivery of the compounds. In these aspects, the compounds are delivered to the lungs of a mammal while inhaling and traverses across the lung epithelial lining to the blood stream.

Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

Some specific examples of commercially available devices suitable for the practice of this invention are, for example and without limitation, the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds. Typically, each formulation is specific to the type of device employed and involves the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, optionally comprise the compounds suspended in water. The formulation also includes, in one aspect, a buffer and a simple sugar (e.g., for protein stabilization and regulation of osmotic pressure). In one embodiment, the nebulizer formulation also contains a surfactant, to reduce or prevent surface induced aggregation of the compounds caused by atomization of the solution in forming the aerosol.

Formulations for use with a metered-dose inhaler device comprise, in one aspect a finely divided powder containing the compounds suspended in a propellant with the aid of a surfactant. The propellant is any conventional material employed for this purpose, such as and without limitation, a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include, without limitation sorbitan trioleate and soya lecithin. Oleic acid is also contemplated as a surfactant in certain aspects.

Formulations for dispensing from a powder inhaler device comprise a finely divided dry powder containing the compound and optionally include a bulking agent, such as and without limitation lactose, sorbitol, sucrose, or mannitol in amounts which facilitate dispersal of the powder from the device, e.g., 50 to 90% by weight of the formulation. In certain embodiments, the compound(s) is/are prepared in particulate form with an average particle size of less than 10 microns, most preferably 0.5 to 5 microns, for most effective delivery to the distal lung.

Nasal delivery of the compounds is also contemplated. Nasal delivery allows the passage of the protein to the blood stream directly after administering the therapeutic product to the nose, without the necessity for deposition of the product in the lung. Formulations for nasal delivery include those with, for example and without limitation, dextran or cyclodextran.

It will be appreciated that in certain aspects, the medicaments of the invention are given as a single dose schedule, or in a multiple dose schedule. A multiple dose schedule is one in which a primary course of delivery for example with 1 to 10 separate doses, is optionally followed by other doses given at subsequent time intervals required to maintain or reinforce the treatment. The dosage regimen is, at least in part, determined by the need of the individual and the judgement of the practitioner.

The invention will now be further described by way of reference only to the following non-limiting examples. It should be understood, however, that the examples following are illustrative only, and should not be taken in any way as a restriction on the generality of the invention described above.

### EXAMPLES

### General Methods

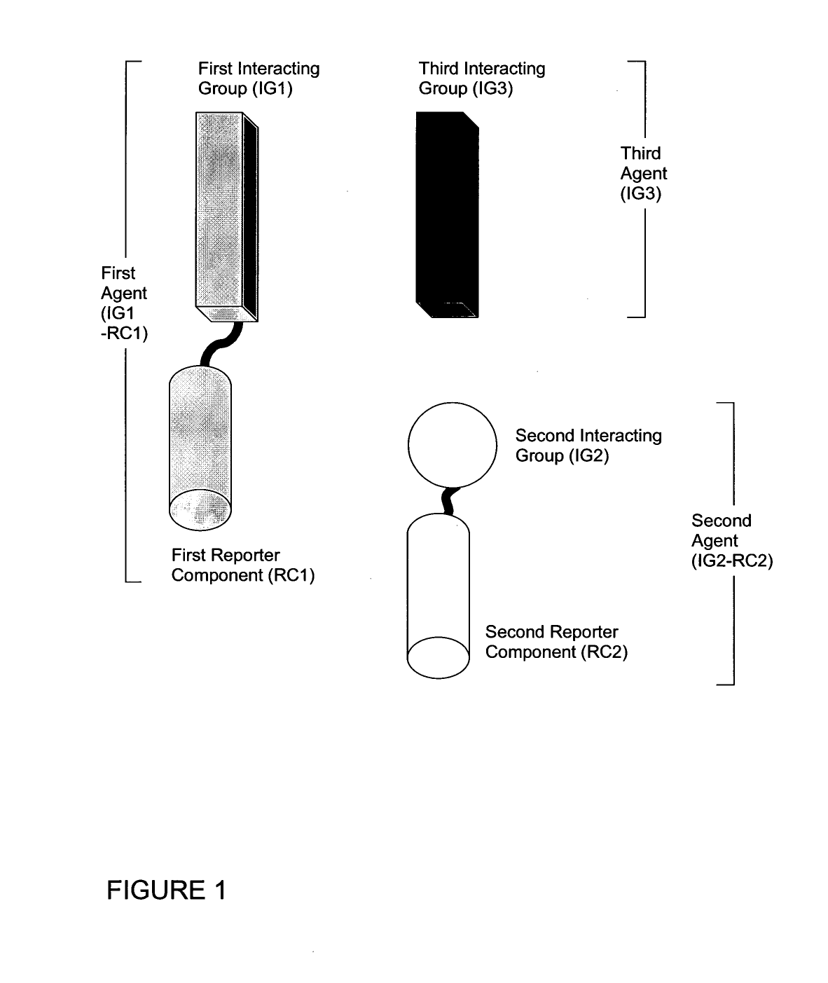

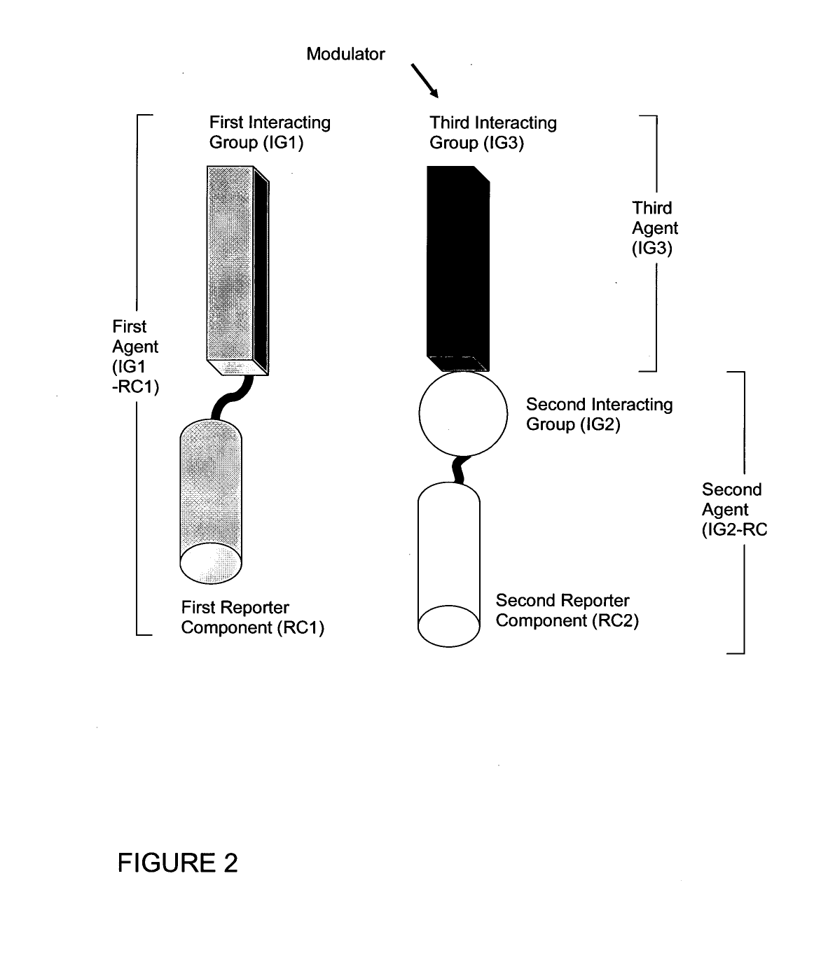

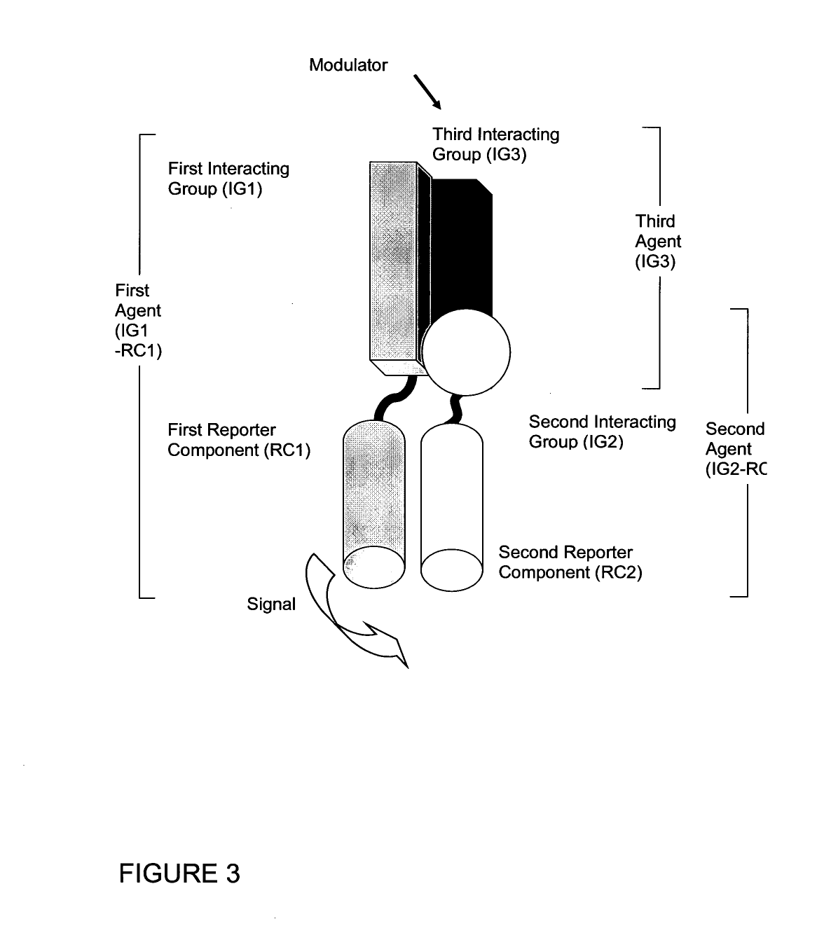

Briefly, referring to FIGS. 1 to 3, the IGs are provided in the form of the two receptors (chemokine receptor and angiotensin receptor). One of the two is attached to an RC (IG1-RC1, IG3). A second IG (IG2-RC2) is derived from a molecule that interacts with the receptors upon ligand binding (e.g. beta-arrestin, or a mutant thereof). The detection system not only detects the formation of the chemokine receptor-angiotensin receptor heterodimer but can distinguish whether a ligand or drug acts as an agonist, partial agonist, antagonist, inverse agonist or partial inverse agonist at the receptor hetero-dimer.

HEK293FT cells were seeded in 6-well plates at a density of approximately 630,000 cells/well and maintained at 37° C., 5% CO₂ in Complete Media (DMEM containing 0.3 mg/ml glutamine, 100 IU/ml penicillin and 100 μg/ml streptomycin (Gibco)) supplemented with 10% fetal calf serum (FCS; Gibco). Transient transfections were carried out 24 h after seeding using GeneJuice (Novagen) according to manufacturer instructions. 24 h post-transfection, cells were washed with PBS, detached using 0.05% trypsin/0.53 mM EDTA, resuspended in HEPES-buffered phenol red free Complete Media containing 5% FCS and added to a poly-L-lysine-coated white 96-well microplate (Nunc). 48 h post-transfection, eBRET assays were carried out following pre-incubation of cells with EnduRen™ (Promega) at a final concentration of 30 μM, at 37° C., 5% CO₂ for 2 h. BRET measurements were taken at 37° C. using the Victor Light plate reader with Wallac 1420 software (Perkin-Elmer). Filtered light emissions were sequentially measured for 2 s in each of the ‘donor wavelength window’ (400-475 nm) and ‘acceptor wavelength window’ (520-540 nm).

The BRET signal observed between interacting proteins is normalized by subtracting the background BRET ratio. This can be done in one of two ways (see Pfleger et al. (2006) Cell Signal 18:1664-1670; Pfleger et al. (2006) Nat Protoc 1:336-344): 1) the ratio of the 520-540 nm emission over the 400-475 nm emission for a cell sample containing only the donor construct is subtracted from the same ratio for a sample containing the interacting acceptor and donor fusion proteins; 2) the ratio of the 520-540 nm emission over the 400-475 nm emission for a cell sample treated with vehicle is subtracted from the same ratio for a second aliquot of the same cell sample treated with ligand. In the following examples, the second calculation will be used and the signal is described as the ‘ligand-induced BRET ratio’.

### Example 1

### Measurement of Detectable Signals Indicative of the Molecular Association of the Chemokine Receptor with the Angiotensin Receptor Using the CCR5-AT1R Heterodimer as an Example

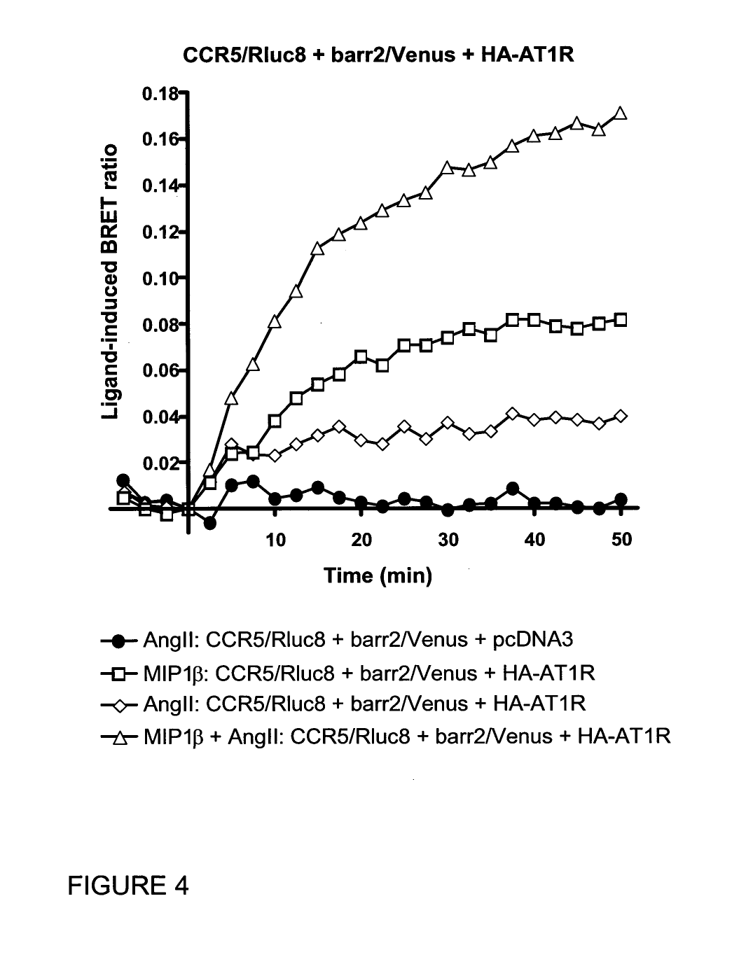

Referring now to FIG. 4, eBRET signals were measured from cells transiently expressing CCR5/Rluc8 and barr2/Venus with HA-AT1R following treatment with either 10⁻⁷M macrophage inflammatory protein 1beta (MIP1β) or 10⁻⁶M angiotensin II (AngII) only or both MIP1β and AngII combined. In parallel, eBRET signals were measured from cells transiently expressing CCR5/Rluc8 and barr2/Venus with pcDNA3 following treatment with 10⁻⁶M angiotensin II (AngII).

Prior to ligand treatment (added at 0 minutes), a baseline eBRET signal was recorded for each of the combinations. AngII treatment of cells expressing CCR5/Rluc8 and barr2/Venus with pcDNA3 did not result in an increase in ligand-induced BRET ratio. MIP1β treatment of cells co-expressing CCR5/Rluc8, barr2/Venus and HA-AT1R resulted in the eBRET signal reaching a peak of about 0.08. A signal was also observed following AngII treatment of cells co-expressing CCR5/Rluc8, barr2/Venus and HA-AT1R. This signal reached approximately 0.04. Treatment of cells co-expressing CCR5/Rluc8, barr2/Venus and HA-AT1R with both MIP1β and AngII resulted in a peak signal of about 0.17, substantially greater than that observed following addition of MIP1β or AngII alone.

This example demonstrates that a signal resulting from the proximity of RC1 and RC2 is detected specifically for the combination where the CC chemokine receptor 5 (CCR5) is IG1, Rluc8 is RC1, beta-arrestin 2 (barr2) is IG2, Venus is RC2 and hemagglutin epitope-tagged AT1R (HA-AT1R) is IG3, and when the modulator, in this case AngII, modulates the association of IG2 and IG3 as a result of interacting specifically with IG3.

This example demonstrates that the inventors have identified the molecular association of the chemokine receptor with the angiotensin receptor.

This example also demonstrates the greater than additive effect of combined treatment with IG1 ligand (MIP1β) and IG3 ligand (AngII; modulator). This provides further and distinct evidence for the molecular association of the chemokine receptor with the angiotensin receptor, as this greater than additive effect is indicative of RC1 and RC2 proximity as a result of IG1-IG2 association in addition to IG2-IG3-IG1 association. This provides evidence against signals originating from non-specific IG1-IG2 association in the absence of an IG1-specific ligand. Without wishing to be bound by theory, this greater than additive effect may also be partly due to IG1 ligand acting as a modulator to modulate the association of IG2 and IG3 via allosteric effects on IG3. Furthermore, this greater than additive effect may also be partly due to an active IG conformation (one that is bound to agonist) being more favourable for signal generation, perhaps enabling increased proximity of RC1 and RC2, or more favourable relative orientation of RC1 and RC2.

This example also demonstrates that IG3 can be tagged, such as by the addition of a hemagglutin (HA) epitope-tag, however, this tag does not constitute a reporter component and does not interfere with and/or contribute to the signal generated by the proximity of RC1 and RC2. Such tagging enables additional information to be ascertained, such as the relative expression level of IG3.

### Example 2

### Measurement of Detectable Signals Indicative of the Molecular Association of the Chemokine Receptor with the Angiotensin Receptor Using the CXCR2-AT1R Heterodimer as an Example

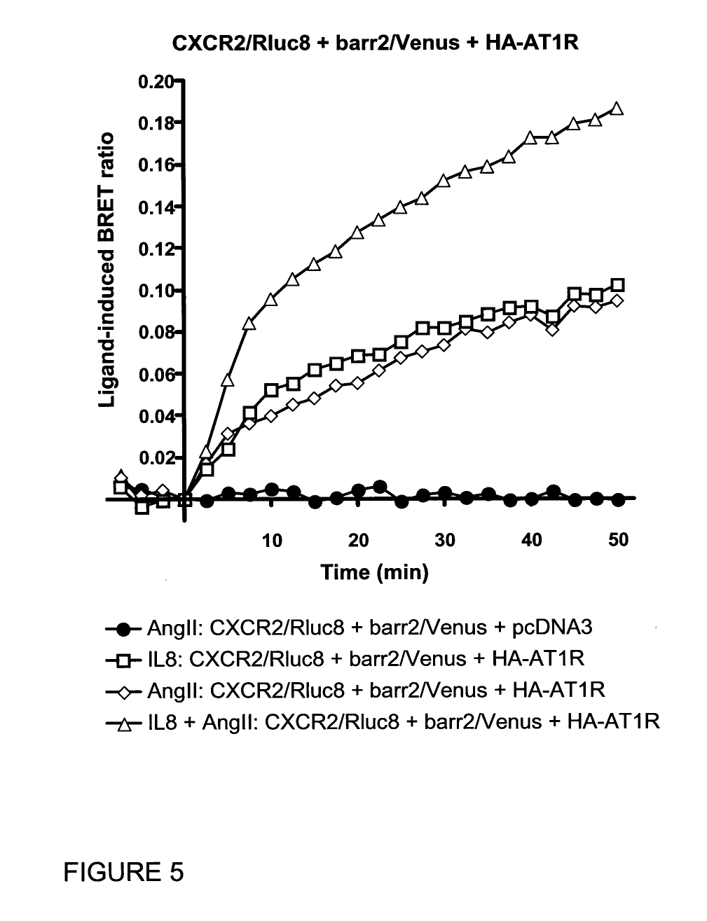

Referring now to FIG. 5, eBRET signals were measured from cells transiently expressing CXCR2/Rluc8 and barr2/Venus with HA-AT1R following treatment with either 10⁻⁷M interleukin 8 (IL8) or 10⁻⁶M angiotensin II (AngII) only or both IL8 and AngII combined. In parallel, eBRET signals were measured from cells transiently expressing CXCR2/Rluc8 and barr2/Venus with pcDNA3 following treatment with 10⁻⁶M angiotensin II (AngII).

Prior to ligand treatment (added at 0 minutes), a baseline eBRET signal was recorded for each of the combinations. AngII treatment of cells expressing CXCR2/Rluc8 and barr2/Venus with pcDNA3 did not result in an increase in ligand-induced BRET ratio. IL8 treatment of cells co-expressing CXCR2/Rluc8, barr2/Venus and HA-AT1R resulted in the eBRET signal reaching a peak of about 0.10. A signal was also observed following AngII treatment of cells co-expressing CXCR2/Rluc8, barr2/Venus and HA-AT1R. This signal reached approximately 0.09. Treatment of cells co-expressing CXCR2/Rluc8, barr2/Venus and HA-AT1R with both IL8 and AngII resulted in a peak signal of about 0.19, substantially greater than that observed following addition of IL8 or AngII alone.

This example demonstrates that a signal resulting from the proximity of RC1 and RC2 is detected specifically for the combination where the CXC chemokine receptor 2 (CXCR2) is IG1, Rluc8 is RC1, beta-arrestin 2 (barr2) is IG2, Venus is RC2 and hemagglutin epitope-tagged AT1R (HA-AT1R) is IG3, and when the modulator, in this case AngII, modulates the association of IG2 and IG3 as a result of interacting specifically with IG3.

This example again demonstrates that the inventors have identified the molecular association of the chemokine receptor with the angiotensin receptor.

This example also demonstrates the additive effect of combined treatment with IG1 ligand (IL8) and IG3 ligand (AngII; modulator). This provides further and distinct evidence for the molecular association of the chemokine receptor with the angiotensin receptor, as this additive effect is indicative of RC1 and RC2 proximity as a result of IG1-IG2 association in addition to IG2-IG3-IG1 association. This provides evidence against signals originating from non-specific IG1-IG2 association in the absence of an IG1-specific ligand. Without wishing to be bound by theory, this additive effect may also be partly due to IG1 ligand acting as a modulator to modulate the association of IG2 and IG3 via allosteric effects on IG3. Furthermore, this additive effect may also be partly due to an active IG conformation (one that is bound to agonist) being more favourable for signal generation, perhaps enabling increased proximity of RC1 and RC2, or more favourable relative orientation of RC1 and RC2.

This example also further demonstrates that IG3 can be tagged, such as by the addition of a hemagglutin (HA) epitope-tag, however, this tag does not constitute a reporter component and does not interfere with and/or contribute to the signal generated by the proximity of RC1 and RC2. Such tagging enables additional information to be ascertained, such as the relative expression level of IG3.

### Example 3

### Measurement of Detectable Signals Indicative of the Molecular Association of the Chemokine Receptor with the Angiotensin Receptor Using the CXCR4-AT1R Heterodimer as an Example

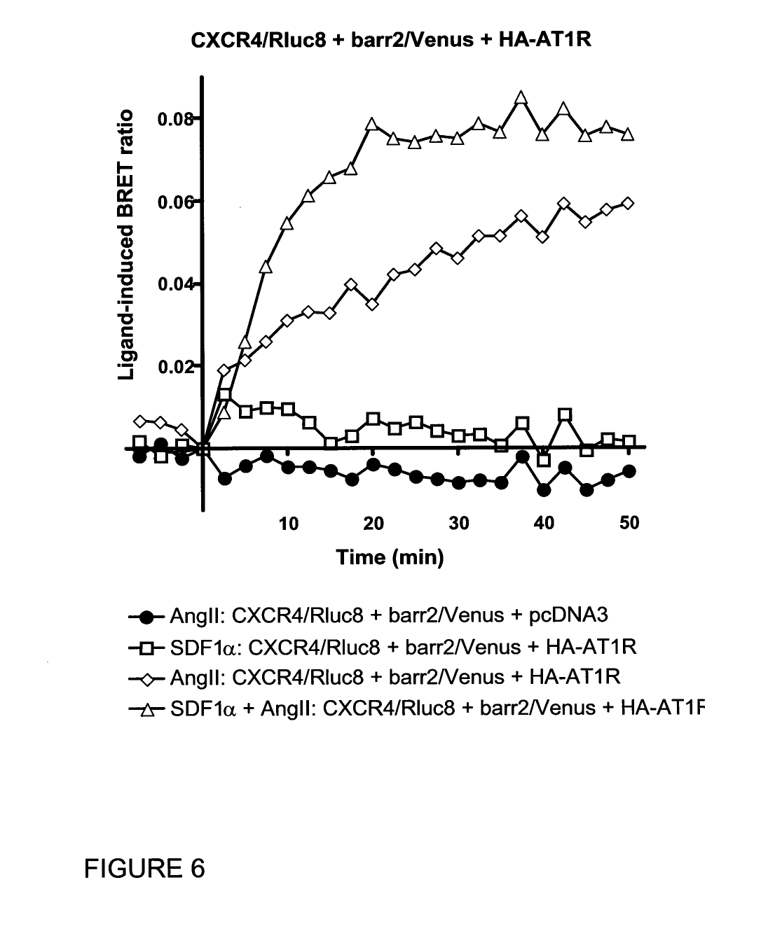

Referring now to FIG. 6, eBRET signals were measured from cells transiently expressing CXCR4/Rluc8 and barr2/Venus with HA-AT1R following treatment with either 10⁻⁷M stromal-cell derived factor-1α (SDF1α) or 10⁻⁶M angiotensin II (AngII) only or both SDF1α and AngII combined. In parallel, eBRET signals were measured from cells transiently expressing CXCR4/Rluc8 and barr2/Venus with pcDNA3 following treatment with 10⁻⁶M angiotensin II (AngII).

Prior to ligand treatment (added at 0 minutes), a baseline eBRET signal was recorded for each of the combinations. AngII treatment of cells expressing CXCR4/Rluc8 and barr2/Venus with pcDNA3 did not result in an increase in ligand-induced BRET ratio. SDF1α treatment of cells co-expressing CXCR4/Rluc8, barr2/Venus and HA-AT1R resulted in a transient eBRET signal reaching a peak of about 0.01. A signal was also observed following AngII treatment of cells co-expressing CXCR4/Rluc8, barr2/Venus and HA-AT1R. This signal reached approximately 0.06. Treatment of cells co-expressing CXCR4/Rluc8, barr2/Venus and HA-AT1R with both SDF1α and AngII resulted in a peak signal of about 0.08, substantially greater than that observed following addition of SDF1α or AngII alone.

This example demonstrates that a signal resulting from the proximity of RC1 and RC2 is detected specifically for the combination where the CXC chemokine receptor 2 (CXCR4) is IG1, Rluc8 is RC1, beta-arrestin 2 (barr2) is IG2, Venus is RC2 and hemagglutin epitope-tagged AT1R (HA-AT1R) is IG3, and when the modulator, in this case AngII, modulates the association of IG2 and IG3 as a result of interacting specifically with IG3.

This example again demonstrates that the inventors have identified the molecular association of the chemokine receptor with the angiotensin receptor.

This example also further demonstrates the greater than additive effect of combined treatment with IG1 ligand (SDF1α) and IG3 ligand (AngII; modulator). This provides further and distinct evidence for the molecular association of the chemokine receptor with the angiotensin receptor, as this greater than additive effect is indicative of RC1 and RC2 proximity as a result of IG1-IG2 association in addition to IG2-IG3-IG1 association. This provides evidence against signals originating from non-specific IG1-IG2 association in the absence of an IG1-specific ligand. Without wishing to be bound by theory, this greater than additive effect may also be partly due to IG1 ligand acting as a modulator to modulate the association of IG2 and IG3 via allosteric effects on IG3. Furthermore, this greater than additive effect may also be partly due to an active IG conformation (one that is bound to agonist) being more favourable for signal generation, perhaps enabling increased proximity of RC1 and RC2, or more favourable relative orientation of RC1 and RC2.

This example also further demonstrates that IG3 can be tagged, such as by the addition of a hemagglutin (HA) epitope-tag, however, this tag does not constitute a reporter component and does not interfere with and/or contribute to the signal generated by the proximity of RC1 and RC2. Such tagging enables additional information to be ascertained, such as the relative expression level of IG3.

### Example 4

### Measurement of a Detectable Signal Indicative of the Molecular Association of the Chemokine Receptor with the Angiotensin Receptor Using the CCR2-AT1R Heterodimer as an Example

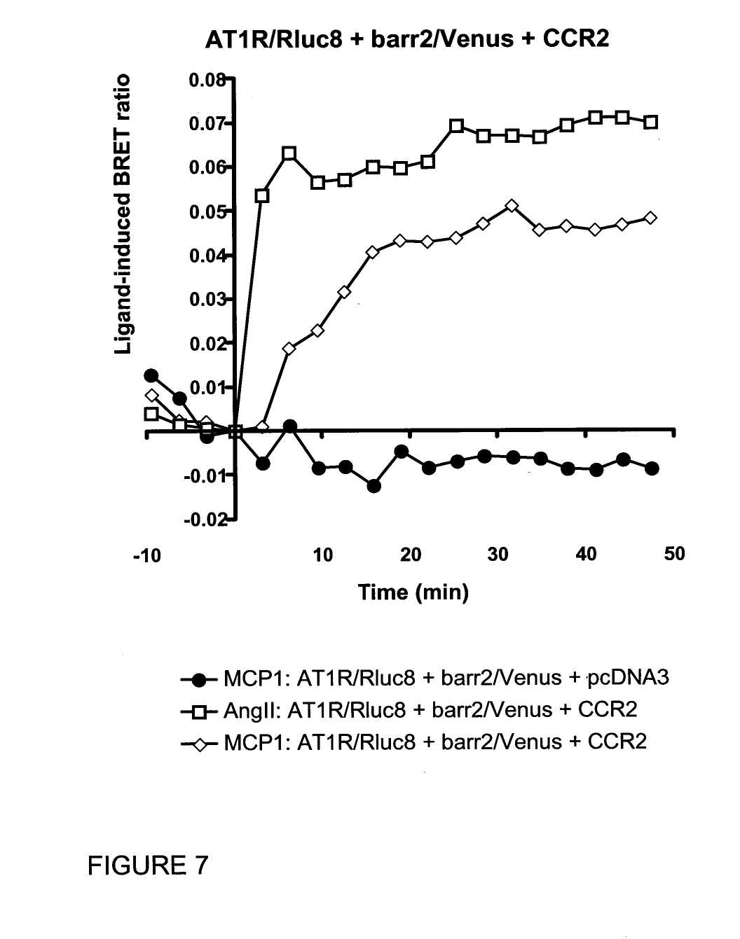

Referring now to FIG. 7, eBRET signals were measured from cells transiently expressing AT1R/Rluc8 and barr2/Venus with CCR2 following treatment with either 10⁻⁷M monocyte chemotactic protein-1 (MCP1) or 10⁻⁶M angiotensin II (AngII). In parallel, eBRET signals were measured from cells transiently expressing AT1R/Rluc8 and barr2/Venus with pcDNA3 following treatment with 10⁻⁷M monocyte chemotactic protein-1 (MCP1).

Prior to ligand treatment (added at 0 minutes), a baseline eBRET signal was recorded for each of the combinations. MCP1 treatment of cells expressing AT1R/Rluc8 and barr2/Venus with pcDNA3 did not result in an increase in ligand-induced BRET ratio. AngII treatment of cells co-expressing AT1R/Rluc8, barr2/Venus and CCR2 resulted in the eBRET signal reaching a peak of about 0.07. A signal was also observed following MCP1 treatment of cells co-expressing AT1R/Rluc8, barr2/Venus and CCR2. This signal reached approximately 0.05.

This example demonstrates that a signal resulting from the proximity of RC1 and RC2 is detected specifically for the combination where the angiotensin receptor 1 (AT1R) is IG1, Rluc8 is RC1, beta-arrestin 2 (barr2) is IG2, Venus is RC2 and CC chemokine receptor 2 (CCR2) is IG3, and when the modulator, in this case MCP1, modulates the association of IG2 and IG3 as a result of interacting specifically with IG3.

This example again demonstrates that the inventors have identified the molecular association of the chemokine receptor with the angiotensin receptor.

### Example 5

### Measurement of a Detectable Signal Indicative of the Molecular Association of the Chemokine Receptor with the Angiotensin Receptor Using the CCR2-AT2R Heterodimer as an Example

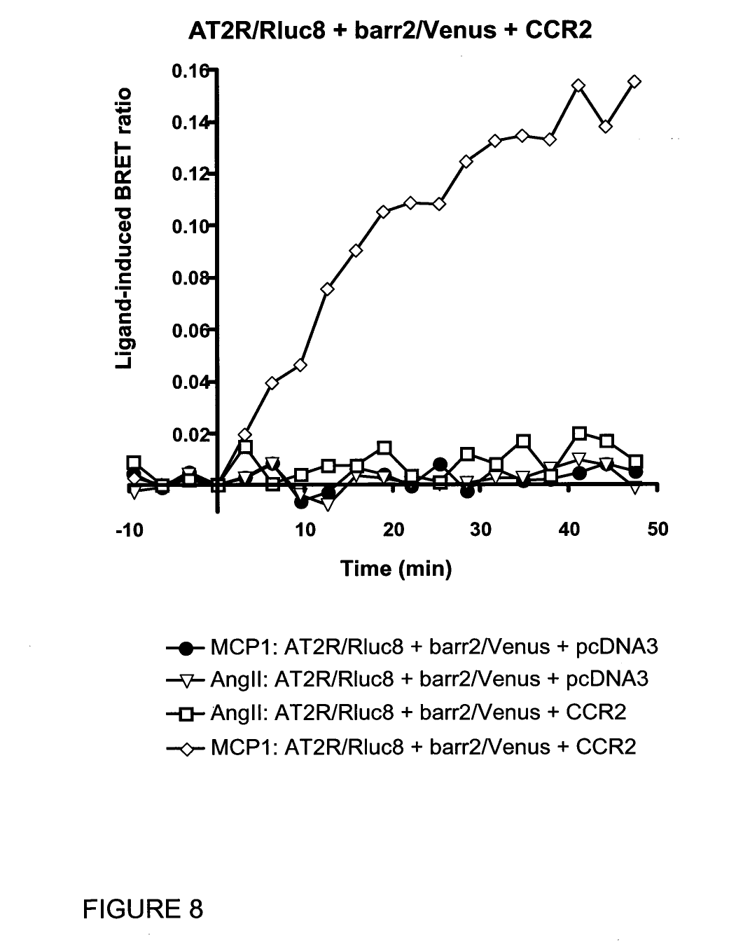

Referring now to FIG. 8, eBRET signals were measured from cells transiently expressing AT2R/Rluc8 and barr2/Venus with CCR2 following treatment with either 10⁻⁷M monocyte chemotactic protein-1 (MCP1) or 10⁻⁶M angiotensin II (AngII). In parallel, eBRET signals were measured from cells transiently expressing AT2R/Rluc8 and barr2/Venus with pcDNA3 following treatment with either 10⁻⁷M monocyte chemotactic protein-1 (MCP1) or 10⁻⁶M angiotensin II (AngII).

Prior to ligand treatment (added at 0 minutes), a baseline eBRET signal was recorded for each of the combinations. MCP1 treatment of cells expressing AT2R/Rluc8 and barr2/Venus with pcDNA3 did not result in an increase in ligand-induced BRET ratio. Furthermore, AngII treatment of cells expressing AT2R/Rluc8 and barr2/Venus with pcDNA3 also did not result in an increase in ligand-induced BRET ratio consistent with AT2R not interacting with barr2 (Turu, G. et al. (2006) Differential beta-arrestin binding of AT1 and AT2 angiotensin receptors. FEBS Letters 580:41-45). Similarly, AngII treatment of cells co-expressing AT2R/Rluc8, barr2/Venus and CCR2 did not result in an increase in ligand-induced BRET ratio. However, a signal was observed following MCP1 treatment of cells co-expressing AT2R/Rluc8, barr2/Venus and CCR2. This signal reached approximately 0.16 after about 50 minutes.

This example demonstrates that a signal resulting from the proximity of RC1 and RC2 is detected specifically for the combination where the angiotensin receptor 2 (AT2R) is IG1, Rluc8 is RC1, beta-arrestin 2 (barr2) is IG2, Venus is RC2 and CC chemokine receptor 2 (CCR2) is IG3, and when the modulator, in this case MCP1, modulates the association of IG2 and IG3 as a result of interacting specifically with IG3.

This example again demonstrates that the inventors have identified the molecular association of the chemokine receptor with the angiotensin receptor.

This example further demonstrates that a detectable signal can be generated when IG1 does not interact with IG2. The lack of signal observed in FIG. 8 upon AngII treatment of cells co-expressing AT2R/Rluc8, barr2/Venus and CCR2 confirms that the signal observed upon MCP1 treatment of this agent combination is not due to IG1-IG2 association.

Therefore, this example provides further and distinct evidence for the molecular association of the chemokine receptor with the angiotensin receptor, as the inability of IG1 to interact with IG2 is indicative of RC1 and RC2 proximity as a result of IG2-IG3-IG1 association and not IG1-IG2 association. This provides further evidence against signals originating from non-specific IG1-IG2 association in the absence of an IG1-specific ligand.

Furthermore, this example demonstrates that the signal results from IG2-IG3-IG1 association as opposed to IG3 activation causing transactivation of IG1, which then associates with IG2, thereby bringing RC1 and RC2 into close proximity without IG2 and IG3 associating.

The claims defining the invention are as follows:

1. A hetero-dimeric or hetero-oligomeric receptor, comprising at least one chemokine receptor subunit associated with at least one angiotensin receptor subunit.

2. A method for the treatment of a patient suffering from an angiotensin-related ailment by administering a therapeutically effective amount of a chemokine receptor-related compound.

3. A method according to claim 2 characterised in that the chemokine receptor-related compound is selective for the chemokine receptor relative to the angiotensin receptor.

4. A method for the treatment of a patient suffering from a chemokine-related ailment by administering a therapeutically effective amount of an angiotensin receptor-related compound.

5. A method according to claim 4 characterised in that the angiotensin receptor-related compound is selective for the angiotensin receptor relative to the chemokine receptor.

6. A method for screening a test compound for potential therapeutic activity against an angiotensin-related ailment using a detector capable of detecting changes in receptor activity, the method comprising the steps of:
a) exposing the test compound to a chemokine receptor;
b) assessing whether and/or the extent to which the activity of the chemokine receptor is modulated compared to activity of the chemokine receptor in the absence of the test compound;
said modulation being indicative of potential therapeutic activity against the angiotensin-related ailment.

7. A method for screening a test compound for potential therapeutic activity against a chemokine-related ailment using a detector capable of detecting changes in receptor activity, the method comprising the steps of:
a) exposing the test compound to an angiotensin receptor;
b) assessing whether and/or the extent to which the activity of the angiotensin receptor is modulated compared to activity of the angiotensin receptor in the absence of the test compound;
said modulation being indicative of potential therapeutic activity against the chemokine-related ailment.

8. A method for screening a test compound for chemokine receptor/angiotensin receptor hetero-dimer/-oligomer selective activity using a detector capable of detecting changes in receptor activity, the method comprising the step of:
determining whether, and/or the extent to which, the test compound interacts with the angiotensin receptor while the angiotensin receptor is associated with the chemokine receptor compared to whether, and/or the extent to which the test compound interacts with the angiotensin receptor in the absence of the chemokine receptor;
such that a test compound that exhibits greater affinity and/or potency and/or efficacy when interacting with the angiotensin receptor while the angiotensin receptor is associated with the chemokine receptor is selective for the chemokine receptor/angiotensin receptor hetero-dimer/-oligomer.

9. A method for screening a test compound for chemokine receptor/angiotensin receptor hetero-dimer/-oligomer selective activity using a detector capable of detecting changes in receptor activity, the method comprising the step of:
determining whether, and/or the extent to which, the test compound interacts with the chemokine receptor while the chemokine receptor is associated with the angiotensin receptor compared to whether, and/or the extent to which the test compound interacts with the chemokine receptor in the absence of the angiotensin receptor;
such that a test compound that exhibits greater affinity and/or potency and/or efficacy when interacting with the chemokine receptor while the chemokine receptor is associated with the angiotensin receptor is selective for the chemokine receptor/angiotensin receptor hetero-dimer/-oligomer.

10. A method for screening a test compound for chemokine receptor/angiotensin receptor hetero-dimer/-oligomer selective antagonism or selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
i). a first agent, comprising the angiotensin receptor coupled to a first reporter component;
ii). a second agent, comprising an interacting group coupled to a second reporter component;
iii). a third agent, comprising the chemokine receptor;
iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting a decrease in the signal as a determination of whether and/or the extent to which the test compound is an antagonist, partial agonist or negative allosteric modulator of the chemokine receptor/angiotensin receptor hetero-dimer/-oligomer and optionally;
c) determining whether, or the extent to which the test compound is an antagonist, partial agonist or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

11. A method for screening a test compound for chemokine receptor/angiotensin receptor hetero-dimer/-oligomer selective antagonism, selective partial agonism or selective negative allosteric modulation using a detector capable of detecting changes in receptor activity, the method comprising the steps of:
a) determining whether, and/or the extent to which, the test compound is an antagonist, partial agonist or negative allosteric modulator of the chemokine receptor/angiotensin receptor hetero-dimer/-oligomer, by contacting said test compound with a system comprising:
i). a first agent, comprising the chemokine receptor coupled to a first reporter component;
ii). a second agent, comprising an interacting group coupled to a second reporter component;
iii). a third agent, comprising the angiotensin receptor;
iv). an agonist of the angiotensin receptor, the chemokine receptor and/or the chemokine receptor/angiotensin receptor hetero-dimer/-oligomer;
wherein proximity of the first and second reporter components generates a signal; and wherein the modulator modulates the association of the interacting group with the angiotensin receptor;
b). detecting a decrease in the signal as a determination of whether and/or the extent to which the test compound is an antagonist, partial agonist or negative allosteric modulator of the chemokine receptor/angiotensin receptor hetero-dimer/-oligomer and optionally;
c) determining whether, or the extent to which the test compound is an antagonist, partial agonist or negative allosteric modulator of the chemokine receptor in the absence of the angiotensin receptor and the angiotensin receptor in the absence of the chemokine receptor; such that a test compound that exhibits greater antagonistic, partial agonistic or negative allosteric modulator properties when interacting with the chemokine receptor/angiotensin receptor hetero-dimer/-oligomer is selective for the chemokine receptor/angiotensin receptor hetero-dimer/-oligomer.

12. A method for screening a test compound for chemokine receptor/angiotensin receptor hetero-dimer/-oligomer selective inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
i). a first agent, comprising the angiotensin receptor coupled to a first reporter component;
ii). a second agent, comprising an interacting group coupled to a second reporter component;
iii). a third agent, comprising a constitutively active chemokine receptor;
wherein proximity of the first and second reporter components generates a signal; and wherein the modulator modulates the association of the interacting group with the chemokine receptor;
b) detecting a decrease in the signal as a determination of whether and/or the extent to which the test compound is an inverse agonist of the chemokine receptor/angiotensin receptor hetero-dimer/-oligomer and optionally;
c) determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine/angiotensin receptor hetero-dimer/-oligomer.

13. A method for screening a test compound for chemokine receptor/angiotensin receptor hetero-dimer/-oligomer inverse agonism using a detector capable of detecting changes in receptor activity, the method comprising the steps of:
a) determining whether, and/or the extent to which, the test compound is an inverse agonist of the chemokine receptor/angiotensin receptor hetero-dimer/-oligomer, by contacting said test compound with a system comprising:
i). a first agent, comprising the chemokine receptor coupled to a first reporter component;
ii). a second agent, comprising an interacting group coupled to a second reporter component;
iii). a third agent, comprising a constitutively active angiotensin receptor;
wherein proximity of the first and second reporter components generates a signal; and wherein the modulator modulates the association of the interacting group with the angiotensin receptor;
b) detecting a decrease in the signal as a determination of whether and/or the extent to which the test compound is an inverse agonist of the chemokine receptor/angiotensin receptor hetero-dimer/-oligomer and optionally;
c) determining whether, or the extent to which the test compound is an inverse agonist of the chemokine receptor in the absence of the angiotensin receptor and the angiotensin receptor in the absence of the chemokine receptor; such that a test compound that exhibits greater inverse agonistic properties when interacting with the chemokine receptor/angiotensin receptor hetero-dimer/-oligomer is selective for the chemokine receptor/angiotensin receptor hetero-dimer/-oligomer.

14. A method for screening a test compound for chemokine receptor/angiotensin receptor hetero-dimer/-oligomer selective positive allosteric modulation using a detector capable of detecting changes in receptor activity, the method comprising the steps of:
a) determining whether and/or the extent to which the test compound is a positive allosteric modulator of the chemokine receptor/angiotensin receptor hetero-dimer/-oligomer, by contacting said test compound with a system comprising:
i). a first agent, comprising the angiotensin receptor coupled to a first reporter component;
ii). a second agent, comprising an interacting group coupled to a second reporter component;
iii). a third agent, comprising the chemokine receptor;
iv). an agonist of the angiotensin receptor, the chemokine receptor and/or the chemokine receptor/angiotensin receptor hetero-dimer/-oligomer; wherein proximity of the first and second reporter components generates a signal; and wherein the modulator modulates the association of the interacting group with the chemokine receptor;
b) detecting an increase in the signal as a determination of whether and/or the extent to which the test compound is a positive allosteric modulator of the chemokine receptor/angiotensin receptor hetero-dimer/-oligomer and optionally;
c) determining whether and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine/angiotensin receptor hetero-dimer/-oligomer.

15. A method for screening a test compound for chemokine receptor/angiotensin receptor hetero-dimer/-oligomer selective positive allosteric modulation using a detector capable of detecting changes in receptor activity, the method comprising the steps of:
a) determining whether and/or the extent to which the test compound is a positive allosteric modulator of the chemokine receptor/angiotensin receptor hetero-dimer/-oligomer, by contacting said test compound with a system comprising:
i). a first agent, comprising the chemokine receptor coupled to a first reporter component;
ii). a second agent, comprising an interacting group coupled to a second reporter component;
iii). a third agent, comprising the angiotensin receptor;
iv). an agonist of the angiotensin receptor, the chemokine receptor and/or the chemokine receptor/angiotensin receptor hetero-dimer/-oligomer;
wherein proximity of the first and second reporter components generates a signal; and wherein the modulator modulates the association of the interacting group with the angiotensin receptor;
b) detecting an increase in the signal as a determination of whether and/or the extent to which the test compound is a positive allosteric modulator of the chemokine receptor/angiotensin receptor hetero-dimer/-oligomer and optionally;
c) determining whether and/or the extent to which the test compound is a positive allosteric modulator of the chemokine receptor in the absence of the angiotensin receptor and the angiotensin receptor in the absence of the chemokine receptor; such that a test compound that exhibits greater positive allosteric modulator properties when interacting with the chemokine receptor/angiotensin receptor hetero-dimer/-oligomer is selective for the chemokine receptor/angiotensin receptor hetero-dimer/-oligomer.

16. Selective agonists and/or selective antagonists and/or selective inverse agonists and/or selective allosteric modulators of the chemokine receptor/angiotensin receptor hetero-dimer/-oligomer.

17. A cell, or fraction of a cell, in which both a chemokine receptor and an angiotensin receptor are over-expressed.

18. A cell, or fraction of a cell, in which a chemokine receptor is over-expressed with an endogenously expressed angiotensin receptor.

19. A cell, or fraction of a cell, in which an angiotensin receptor is over-expressed with an endogenously expressed chemokine receptor.
